(19) United States
(12) Patent Application Publication
(10) Pub. No.: US 2004/0115198 A1
(43) Pub. Date: Jun. 17, 2004

(54) Activation of lymphocyte populations expressing NKG2D using anti-NKG2D antibodies and ligand derivatives

(75) Inventors: Thomas Spies, Seattle, WA (US); Veronika Spies, Seattle, WA (US)

Correspondence Address: Steven L. Highlander, Esq., Fulbright & Jaworski, LLP, Suite 2400, 600 Congress Ave., Austin, TX, 78701, US

(73) Assignee: Fred Hutchinson Cancer Research Center

(21) Appl. No.: 10/648,978

(22) Filed: Aug. 27, 2003

Related U.S. Application Data
(63) Continuation of application No. PCT/US02/05343, filed on Feb. 21, 2002.
(60) Provisional application No. 60/272,406, filed on Feb. 28, 2001.

(51) Int. Cl. A61K 39/395

(52) U.S. Cl. 424/145.1

(57) ABSTRACT

The present invention provides various methods for stimulating a cell expressing an NKG2D receptor, including artificially engineered cell populations. Provided, in accordance with the invention. are monoclonal antibodies that bind to NKG2D extracellular domains and facilitate the interaction of other NKG2D domains with DAP10. Of particular interest are treating cancers and viral infections, and the stimulation, both in vivo and ex vivo, of cytokine secretion.

### BACKGROUND OF THE INVENTION

The government owns rights in the present invention pursuant to grant numbers RO1 AI30581 and POI CA18221 from the National Institutes of Health.

1. Field of the Invention

The present invention relates generally to the field of immunology. More particularly, it describes stimulation of immune functions through cell surface molecules known as NKG2D, which may be targeted to treat cancer, viral diseases and other conditions.

2. Description of Related Art

Intracellular antigens, such as viral proteins, are recognized by CD8 αβ T-cells after they are processed to short peptides and presented by polymorphic major histocompatibility complex (MHC) class I molecules (Germain & Margulies, 1993). T-cells become activated by engagement of their clonotypic T-cell antigen receptor (TCR)-CD3 complexes by specific MHC class I-peptide molecules and of the costimulatory CD28 receptor by its CD80-CD86 ligands, which are expressed on professional antigen-presenting cells (Davis et al., 1998; Lenschow et al., 1996). Proficient occupation of both receptors results in T-cell proliferation and interleukin (IL)-2 production whereas triggering of the TCR-CD3 complex alone favors T-cell anergy or apoptosis (Hara et al., 1985; Thompson et al., 1989; Gimmi et al., 1991; Linsley et al., 1991; Harding et al., 1992; Gribben et al., 1995; Chambers & Allison, 1999).

In addition to these central receptor-ligand interactions, diverse adhesion or signaling molecules modulate T-cell activation. The latter may include inhibitory or stimulatory receptors that were first identified on natural killer (NK) cells, but are also expressed on T-cells. Among these are isoforms of the killer cell immunoglobulin (Ig)-like receptors (KIR), which interact with MHC class I HLA-A, -B, or -C, and the lectin-like CD94-NKG2A or CD94-NKG2C receptor pairs that bind HLA-E (Ravetch & Lanier, 2000; Lee et al., 1998). The inhibitory receptors have cytoplasmic immunoreceptor tyrosine-based inhibitory motifs (ITIM) that function by recruitment of tyrosine phosphatases (Long, 1999). Activating isoforms of KIR, which lack ITIM, and the CD94-NKG2C receptor associate with an adaptor protein, DAP12, which signals similar to the CD3ζ chain, by activation of tyrosine kinases after phosphorylation of its tyrosine-based activation motif (ITAM) (Lanier et al., 1998). When NK cells engage target cells, the aggregate effects of signals from these and other receptors become integrated to favor inhibition or activation of effector functions (Lanier, 2000). With T-cells, there is evidence that ligand engagement of inhibitory receptors can increase TCR-dependent activation thresholds (Phillips et al., 1995; Carena et al., 1997; Ikeda et al., 1997; Bakker et al., 1998; Noppen et al., 1998); however, whether and how signals from activating receptors are functionally integrated is unknown.

A stimulatory receptor of particular interest is NKG2D, as it is expressed on most NK cells, CD8 αβ T-cells and γδ T-cells, and thus is the most widely distributed “NK cell receptor” known (Bauer et al., 1999). NKG2D shares no close relationships with other NKG2 family members and is not associated with CD94. It forms homodimers that pair with an adaptor protein, DAP10, which may signal by recruitment of phosphotidylinositol-3 kinase (P13K) upon phosphorylation of a tyrosine-based motif in its cytoplasmic domain (Wu et al., 1999). Whereas the function of KIR and CD94-NKG2 receptors is to monitor the expression of MHC class I molecules, which is often impaired on virus-infected or tumor cells (Ravetch & Lanier, 2000), NKG2D interacts with ligands that are not constitutively but inducibly expressed.

Among these are human MICA and MICB, which are distant homologs of MHC class I, but have no function in antigen presentation (Bahram et al., 1994; Bahram & Spies, 1996; Groh et al., 1996; Li et al., 1999). These molecules are stress-induced similar to heat-shock protein 70 (hsp70), presumably owing to the presence of putative heat-shock elements in the 5′-flanking regions of the corresponding genes (Groh et al., 1996; Groh et al., 1998). They have a restricted tissue distribution in intestinal epithelium and are frequently expressed in epithelial tumors (Groh et al., 1996; Groh et al., 1999). While it is known that engagement of NKG2D by MIC stimulates NK cell and γδ T-cell effector functions, and may positively modulate CD8 αβ T-cell responses (Bauer et al., 1999; Groh et al., 1998), the ability to exploit this knowledge has not been demonstrated.

### SUMMARY OF THE INVENTION

Therefore, in a first embodiment, there is provided a method for expanding a human T-cell population that expresses a natural or engineered NKG2D comprising contacting said population with an NKG2D ligand. The NKG2D ligand may be an anti-NKG2D antibody, or an NKG2D-binding fragment thereof. The contacting may be performed in vivo or ex vivo. The anti-NKG2-D antibody fragment may be Fab, F(ab′)₂, or single-chain antibody.

The cell population may be a CD8⁺ population or a CD4⁺ population, a T cell population, an NK cell population or a monocyte population. Where a T cell population, it may be an antigen-specific T cell population, for example, from a subject with a primed anti-tumor responsor with a primed anti-viral response. The T cell population also may be from an immunocompromised subject. In a further, embodiment, the T cell population may be induced to secrete lymphokines.

In another embodiment, there is provided a method for inducing lymphokine secretion from a human cell population that expresses a natural or engineered comprising contacting said population with an anti-NKG2-D antibody, or an NKG2-D-binding fragment thereof. The lymphokine may be INF-γ, TNF-α, GM-CSF, IL-2 or IL-4.

In still another embodiment, there is provided a method for enhancing an antigen-specific T cell response in a subject comprising (a) obtaining a population of antigen-specific T cells, (b) contacting said population of antigen-specific T cells with an anti-NKG2-D antibody, or an NKG2-D-binding fragment thereof, and (c) administering said population to said subject.

In still yet another embodiment, there is provided a method for treating cancer comprising (a) obtaining a population of antigen-specific T cells from a subject having cancer, (b) contacting said population of antigen-specific T cells with an anti-NKG2-D antibody, or an NKG2-D-binding fragment thereof, and (c) administering said population to said subject. The cancer may be an epithelial tumor, for example, a carcinoma such as a carcinoma of the breast, lung, colon, kidney, prostate, or ovary. The cancer also may be a melanoma.

In a further embodiment, this is provided a method for treating a viral infection comprising (a) obtaining a population of antigen-specific T cells from a subject having a viral infection, (b) contacting said population of antigen-specific T cells with an anti-NKG2-D antibody, or an NKG2-D-binding fragment thereof, and (c) administering said population to said subject.

In still a further embodiment, there is provided a method of stimulating the immune system of an immunocompromised subject comprising (a) obtaining a population of antigen-specific T cells from said subject, (b) contacting said population of antigen-specific T cells with an anti-NKG2-D antibody, or an NKG2-D-binding fragment thereof, and (c) administering said population to said subject.

In yet a further embodiment, there is provided a method of stimulating an effector function a lymphocyte comprising (a) obtaining a population of lymphocytes, and (b) contacting said population of lymphocytes with an anti-NKG2-D antibody, or an NKG2-D-binding fragment thereof.

In an additional embodiment, there is provided a method of stimulating a memory function of a lymphocyte comprising (a) obtaining a population of lymphocytes, and (b) contacting said population of lymphocytes with an anti-NKG2-D antibody, or an NKG2-D-binding fragment thereof.

### BRIEF DESCRIPTION OF THE DRAWINGS

FIGS. 1A & 1B—Induction of MIC expression on CMV-infected fibroblasts and endothelial cells. FIG. 1A. With primary human skin fibroblast cultures infected with CMV AD169, staining with mAb 6D4 and flow cytometry showed substantial increases of MIC expression (filled profiles) between 24 (upper panel) and 72 h (bottom panel) after infection while MHC class I (shaded profiles) detected with mAb W6/32 decreased. Similar results were obtained with a number of different anti-MIC mAbs. Open profiles are Ig-isotype control stainings. FIG. 1B. Two-color immunofluorescence stainings of umbilical vein endothelial cells infected at low multiplicity with CMV VHL/e showed two distinct cell populations with inversely correlated surface levels of MIC and MHC class I.

FIGS. 2A & 2B—Association of induced MIC expression with productive CMV infection in cultured endothelial cells and lung disease. FIG. 2A. Two-color immunostainings of endothelial cell monolayers partially infected with CMV VHL/e for CMV IE-1 (mAb NEA-9221, visualized by green fluorescence with Streptavidin CY conjugate) and MIC (mAb 6D4, visualized by red fluorescence with Streptavidin Alexa 594 conjugate). Nuclei were stained with diamino-phenylindole. See Methods for technical details. FIG. 2B. Cryostat sections of CMV interstitial pneumonia specimens stained for MIC (large micrograph; brown diamino benzidine peroxidase substrate staining) or for MIC and CMV delayed early DNA-binding protein p52 (small insert micrograph; additional Fast Red peroxidase substrate staining). Due to technical limitations, better contrast could not be achieved in the two-color tissue stainings, which serve as a complement to the image shown in FIG. 2A. No stainings were observed with sections of control lung specimens.

FIGS. 3A-F—Augmentation of anti-CMV cytolytic T-cell responses by MICA-NKG2D. FIGS. 3A & 3B. Primary skin fibroblast cultures typed for HLA-A1 or -A2 expressing unaltered versus increased and decreased amounts of MIC and MHC class I 12 and 72 h after infection with CMV AD 169, respectively, were tested as targets for HLA-matched pp65-specific CD28⁻CD8 αβ T-cell clones in chromium release assays. Fluorescence profiles in histograms are labeled according to the time points of MIC or MHC class I antibody staining. Open profiles are Ig-isotype control stainings. FIGS. 3C & 3F. At 12 h post-infection, the cytolytic activities of the T-cell clones 8E8-403 (HLA-A1) and 19D1-66 (HLA-A2) could be inhibited by anti-MHC class I (mAb W6/32) but not by anti-MIC (mAb 6D4) or anti-NKG2D (mAb 1D 11). At 72 h post-infection, mAbs against MIC or NKG2D had inhibitory effects. Similar data were obtained with additional two HLA-A1- and five HLA-A2-restricted T-cell clones (see Methods). FIGS. 3D & 3E. No lysis was scored with HLA-mismatched combinations of T-cells and virus-infected targets. Ranges of standard deviations (SD) are indicated above bars in percent.

FIG. 4—Antigen dose-dependent augmentation of cytolytic T-cell function by NKG2D. Cytotoxic responses of pp65-specific T-cells against C1R-A2-MICA double transfectants pulsed with the HLA-A2-restricted NLVPMVATV peptide were substantially stronger than those against identically treated C1R-A2 transfectants within a range of suboptimal peptide concentrations. These increases were diminished by mAb against MICA or NKG2D. The results obtained with the 4H6-254 T-cell clone were representative of five T-cell clones tested. All assays were done in triplicate with deviations that were not greater than about 3%.

FIGS. 5A-D—Stimulation of T-cell cytokine secretion by NKG2D. C1R-A2-MICA cells pulsed with the specific pp65 peptide stimulated secretion of much larger amounts of (FIG. 5A) IFN-γ, (FIG. 5B) TNF-α, (FIG. 5C) IL-2, and (FIG. 5D) IL-4 by the HLA-A2-restricted pp65-specific T-cell clone 2E9-269 than C1R-A2 cells pulsed with the same peptide concentrations. Note that in the absence of MICA on the stimulator cells no IL-2 was detected in T-cell supernatants. The results shown were similar to those obtained with four other T-cell clones (see Methods) and for GM-CSF and IL-4 (data not shown). Each bar represents the cytokine ELISA read-out from three pooled wells of T-cell supernatants. All of these assays, including parallel experiments with anti-NKG2D, anti-MIC or isotype control antibody (data not shown), were performed three times with comparable results. The total number of data points (bars) was 3240 (12 bars/graph×5 T-cell clones×6 cytokines×3 antibodies×3 experiments).

FIGS. 6A-C—Stimulation by NKG2D of IL-2 production in peripheral blood CMV-specific CD28⁻ CD8 αβ T-cells. FIG. 6A. Among CD8 αβ T-cells isolated by negative selection from peripheral blood, pp65-specific T-cells were identified by fluorescence staining with HLA-A2 tetramers refolded with pp65 peptide and flow cytometry. The gated CD28⁻ population of these T-cells included a proportion of cells that stained positively for intracellular IL-2 after short-term coculture with peptide-pulsed C1R-A2-MICA cells (FIG. 6C) but not after identical coculture with peptide-pulsed C1R-A2 cells lacking MIC (FIG. 6B). See Methods for further technical details.

FIGS. 7A-C—Costimulation by NKG2D of TCR-CD3 complex-dependent IL-2 production and proliferation of CD28^{- CD}8 αβ T-cells. FIG. 7A. Triggering of the T-cell clone 4H6-254, which was representative of five T-cell clones tested, with a range of concentrations of plate-bound anti-CD3 mAb resulted in minimal or modest T-cell proliferation measured by [³H]thymidine incorporation. However, T-cell proliferation was strongly amplified in the additional presence of solid-phase anti-NKG2D (mAb 1D11) but not of Ig-isotype control antibody. FIG. 7B. Combined triggering with anti-CD3 and anti-NKG2D potently induced T-cell IL-2 secretion. Data shown are representative of five T-cell clones tested. FIG. 7C. Anti-NKG2D in combination with anti-CD3 superinduced proliferation of freshly isolated peripheral blood CD28⁻ CD8 αβ T-cells. Experiments in FIG. 7A & 7B were done in triplicate with no more than about 3% deviation.

### DESCRIPTION OF ILLUSTRATIVE EMBODIMENTS

I. Mic Binding to NKG2D

The present invention stems, in part, from the inventors' earlier discoveries of the existence and function of MICA and MICB and of their role as ligands for NKG2D. Herein, the significance of MIC immunobiology is again demonstrated by showing that MIC expression is induced by human cytomegalovirus (CMV) infection, and further, that engagement of the NKG2D receptor by MIC strongly augments anti-CMV CD8 αβ T-cell responses despite the viral interference with antigen presentation. This probably represents an important factor in the immunological control of this virus, which establishes lifelong persistence marked by alternating periods of latency and reactivation in infected hosts, and can likely be extrapolated to at least some other viral and microbial infections.

Notably, a recent report has suggested that a CMV glycoprotein, UL16, which interacts with MIC and a set of cell surface proteins termed ULBP, may interfere with NKG2D function. Posnett et al. (1999). If substantiated, this would lend further support to the hypothesis that MIC-NKG2D may effectively enable the immune system to combat this virus. Moreover, because MIC expression is associated with diverse epithelial tumors including lung, breast, colon, ovary, prostate and renal cell carcinomas (Groh et al., 1999), these results indicate that their interaction with NKG2D may also stimulate responses by CD8 αβ T-cells specific for tumor antigens. Together, these results support the model that the MIC-NKG2D system, with its ability to activate NK cells and T-cells, may function as an emergency defense against infectious agents and hazardous conditions that cause cellular distress.

The NKG2D-mediated augmentation of effector T-cell responses, such as cytotoxicity and secretion of IFN-γ and TNF-α, presumably involves ligand adhesion as indicated by the strong binding of soluble MICA to cell surface NKG2D (Bauer et al. 1999). More significantly, however, NKG2D potently stimulates TCR-CD3 complex-dependent T-cell proliferation and IL-2 production. Thus, NKG2D functions as a costimulatory receptor although its mechanism of signaling via DAP10 may not have been completely resolved. These results highlight the significance of MIC expression throughout the gastrointestinal epithelium (Groh et al., 1996), implying that this site may have costimulatory capacity.

Among peripheral effector CD8 αβ T-cells, about 20-60% are negative for CD28, depending on age and factors such as chronic infections (Posnett et al., 1999). These T-cells have been found hyporesponsive to stimulation by anti-CD3 even in the presence of exogenously added IL-2 (Azuma et al., 1993). The current results show that ligand engagement of NKG2D can reverse this anergic state and rescue autocrine proliferation. This indicates that triggering of NKG2D by suitably engineered derivatives of antibodies or ligands can be applied to effectively expand specific effector CD8 αβ T-cells in vitro and to boost primed T-cell responses by local targeting or systemic administration in vivo. The inventors have previously reported that MICS function as antigens for a subset of γδ T-cells (V_{δ}1 γδ T-cells) that predominates in epithelial sites (Groh et al., 1998; Groh et al., 1999). Thus, the current evidence suggests that, in the activation of these T-cells, MIC may provide signal 1 (TCR-dependent) as well as signal 2 (NKG2D-dependent).

Because of the broad distribution among lymphocyte subsets and functional potency of NKG2D, it appears imperative that the expression of its ligands must be tightly controlled to limit T-cell proliferation and avert autoimmune reactions. By the same token, the substantial expression of MIC on large proportions of gastrointestinal epithelium suggests that NKG2D may be regulated as well to minimize the risk of widespread inflammation. In addition to MICA and MICB, NKG2D interacts with other ligands that have disparate sequences although they share common MHC class I-like α1α2 domains. These include the putative human ULBP proteins and their possible murine counterparts—the retinoic acid early inducible RAE-1 family of ligands (Chalupny et al., 2000; Cerwenka et al., 2000; Diefenbach et al., 2000). As of yet, little is known about the immunologically relevant expression of these molecules, except that they may be present on some tumor cells (Diefenbach et al., 2000).

II. NKG2D

Major histocompatability complex class I molecules are ligands for inhibitory or activating natural killer (NK) cell receptors that are expressed on NK cells and T cells. These include three isoforms of the immunoglobulin (Ig)-like killer cell receptors that interact with HLA-A, -B or -C, and CD94 paired with NKG2A or NKG2C, which bind HLA-E. Engagement of these receptors modulates NK cell responses and TCR-dependent T-cell activation.

In 1999, Bauer et al. identified NKG2D as a receptor for stress-induced MICA. NKG2D had previously been proposed to have an activating function because of the lack of a tyrosine-based inhibitory motif in its cytoplasmic tail. In addition, it was known that NKG2D's partner, DAP10, interacts with the p85 subunit of P13-kinase. The study by Bauer et al. used soluble MICA in binding assays, representational difference analysis (RDA) and protein immunoprecipitation with specific monoclonal antibodies to show that NKG2D is a receptor for MICA. Its apparently molecular mass of 42 kD matched independent data obtained with polyclonal antibodies.

NKG2D lacks a tyrosine-based inhibitory motif in its cytoplasmic tail and may function as an activating receptor; signaling may be enabled by DAP10, which has an SH2 domain-binding site for the p85 subunit of phoshoinositide 3-kinase. An activating function is supported by the inhibition of γδ T-cell recognition of MICA mediated by monoclonal antibody again γδ T-cell receptor. However, these responses can also be inhibited by monoclonal antibodies again γδ T-cell receptors, implying that their activation also requires T-cell receptor engagement.

To examine whether NKG2D can function in the absence of T-cell receptor signaling, Bauer et al. (1999) used NK cell effectors. These showed the expected cytotoxicity against Daudi cells, which lack β₂-microglobulin (β₂m) and thus MHC class I, whereas Daudi-β₂m transfectants were protected by the restored expression of MHC class I; inhibition of KNKL was mediated by HLA-E, the ligand for CD94-NKG2A. However, coexpression of MICA sensitized Daudi-β₂m cells to lysis, which could be inhibited by anti-MICA and anti-NKG2D antibody. MICA did not diminish surface expression of class I. Hence, masking of HLA-E on Daudi-β₂m-MICA cells increased cytolysis to a level above that recorded with Daudi cells. Ligation of NKG2D on NKL with monoclonal antibodies induced redirected lysis of Fc receptor (FcR)-bearing cells, similar to responses with anti-CD16. Thus, in agreement with its broad distribution on most γδ T-cells, CD8⁺ αβ T cells and NK cells, NKG2D has an activating function triggered by engagement of MICA (or presumably of MICB) over a diverse range of effector cells.

DNA sequences for NKG2D can been found in WO 92/17198, incorporated herein by reference. NKG2D genes, and their corresponding cDNA can be inserted into an appropriate cloning vehicle for manipulation thereof. In addition, sequence variants of the polypeptide may be utilized. These may, for instance, be minor sequence variants of the polypeptide that arise due to natural variation within the population or they may be homologes found in other species. They also may be sequences that do not occur naturally but that are sufficiently similar that they function similarly and/or elicit an immune response that cross-reacts with natural forms of the polypeptide. Sequence variants can be prepared by standard methods of site-directed mutagenesis such as those described below in the following section.

A. Variants of NKG2D

Amino acid sequence variants of NKG2D can be substitutional, insertional or deletion variants. Substitutional variants typically contain the exchange of one amino acid for another at one or more sites within the protein, and may be designed to modulate one or more properties of the polypeptide such as stability against proteolytic cleavage. Substitutions preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

Insertional variants include fusion proteins such as those used to allow rapid purification of the polypeptide and also can include hybrid proteins containing sequences from other proteins and polypeptides which are homologues of the polypeptide. For example, an insertional variant could include portions of the amino acid sequence of the polypeptide from one species, together with portions of the homologous polypeptide from another species. Other insertional variants can include those in which additional amino acids are introduced within the coding sequence of the polypeptide These typically are smaller insertions than the fusion proteins described above and are introduced, for example, into a protease cleavage site.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid substitutions can be made in a protein sequence, and its underlying DNA coding sequence, and nevertheless obtain a protein with like properties. It is thus contemplated by the inventors that various changes may be made in the DNA sequences of genes without appreciable loss of their biological utility or activity. Table 1 shows the codons that encode particular amino acids.

In making such changes, the hydropathic index of amino acids may be considered. The importance of the hydropathic amino acid index in conferring interactive biologic function on a protein is generally understood in the art (Kyte & Doolittle, 1982).

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: Isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3);

TABLE 1

| Amino Acids | | | Codons |
|---|---|---|---|
| Alanine | Ala | A | GCA GCC GCG GCU |
| Cysteine | Cys | C | UGC UGU |
| Aspartic acid | Asp | D | GAC GAU |
| Glutamic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like.

Each amino acid has been assigned a hydropathic index on the basis of their hydrophobicity and charge characteristics (Kyte & Doolittle, 1982), these are: Isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e., still obtain a biological functionally equivalent protein. In making such changes, the substitution of amino acids whose hydropathic indices are within ±2 is preferred, those which are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is understood that an amino acid can be substituted for another having a similar hydrophilicity value and still obtain a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

B. Fusion Proteins

Within one embodiment of the invention, specific fusion proteins of NKG2D are contemplated. By fusing the external domain of NKG2D with a distinct DAP10 interacting domain or with cytoplasmic domains derived from other signaling molecules, for example CD28, one may be able to engineer cells that respond to NKG2D ligands and potentially create a system with enhanced signaling capabilities. Alternatively, one may link transmembrane or cytoplasmic domains from NKG2D with distinct extracellular ligand binding domains. This permits “designer” cells to be created that respond to alternative signaling molecules.

III. LIGANDS FOR NKG2D

A. MICA and MICB

MICA and MICB are natural ligands for NKG2D. Although MICA and MICB are encoded by genes in the MHC, they share only about 27% amino acid sequence identity with conventional MIHC class I chains in their extracellular α1α2α3 domains. MICA/B themselves are closely related, sharing 84% identical amino acids (Bahram et al., 1994; Bahram & Spies, 1996). Unlike MHC class I, the highly glycosylated MICA/B surface proteins are not associated β₂-microglobulin and peptides and lack the main CD8 binding site (Groh et al., 1996). The crystal structure MICA revealed a dramatically altered MHC class I fold in which the membrane-distal α1α2 superdomain is flexibly linked to the Ig-like α3 doamin, such that all of its surfaces including the underside of the β-pleated sheet are accessible for potential molecular interactions. The α1α2 helices on top of the β-strand platform are highly distorted and do not form a potential ligand-binding groove (Li et al., 1999). These distortions are similar to those in the mouse nonclassical MHC class I T22 molecule, which has been shown to interact with a small subset of γδ T-cells from murine spleen. Sequences directly related to MICA/B are conserved in the genomes of most, if not all, mammalian species with the possible exception of rodents, and are expressed in all of a number of diverse non-human primates that have been investigated (Bahram et al., 1994).

Unlike MHC class I molecues, which are ubiquitously expressed, the distribution of MICA/B proteins in normal tissues is restricted to intestinal epithelium. Notably, the 5′-end of flanking regions of both genes include putative heat-shock elements similar to those in hsp70 genes (Groh et al., 1996). Heat shock treatment of epithelial cell lines grown under conditions of minimal cell proliferation results in potent increases of MICA/B mRNA and surface protein expression (Groh et al., 1998). Possibly associated with this apparent stress-inducible regulation, MICA/B have been found variably expressed in many, but not all, epithelial tumros including lung, breast, kidney, ovary, prostate and colon carcinomas (Groh et al. 1999).

B. Other Natural Ligands

Several other binding ligands for NKG2D include the human ULBP proteins and their possible murine counterparts—the retinoic acid early inducible RAE-1 family of ligands (Chalupny et al., 2000; Cerwenka et al., 2000; Diefenbach et al., 2000. These molecules, or fragments or derivatives thereof, may be used to stimulate NKG2D in a fashion analogous to MICA/B.

C. Antibodies The present inventors have successfully produced monoclonal antibodies that bind specifically to NKG2D. In particular, the antibodies ID11 (ATCC Deposit No. PTA-3056, deposited Feb. 15, 2001) and 5C6 (ATCC Deposit No. PTA-3055, deposited Feb. 15, 2061) are suitable for all of the disclosed methods. Polyclonal antibodies and other monoclonal antibodies may be produced that may be utilized according to the present invention. For therapeutic purposes, antibodies may be humanized and/or otherwise manipulated to optimize efficacy.

D. Mimetics

In addition to the biological functional equivalents discussed above, the present inventors also contemplate that structurally similar compounds may be formulated to mimic the key portions of peptide or polypeptides of the present invention. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turn within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a β sheet and an alpha helix bridged in the interior core by three disulfides.

Beta II turns have been mimicked successfully using cyclic L-pentapeptides and those with D-amino acids (Weisshoff et al., 1999). Also, Johannesson et al. (1999) report on bicyclic tripeptides with reverse turn inducing properties. Methods for generating specific structures have been disclosed in the art. For example, alpha-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. Beta-turns permit changed side substituents without having changes in corresponding backbone conformation, and have appropriate termini for incorporation into peptides by standard synthesis procedures. Other types of mimetic turns include reverse and gamma turns. Reverse turn mimetics are disclosed in U.S. Pat. Nos. 5,475,085 and 5,929,237, and gamma turn mimetics are described in U.S. Pat. Nos. 5,672,681 and 5,674,976.

E. Purification of Protein Ligands

In most embodiments, purification of protein ligands for use according to the present invention will be required. Generally, “purified” will refer to a protein or peptide composition that has been subjected to fractionation to remove various other components, and which composition substantially retains its expressed biological activity. Where the term “substantially purified” is used, this designation will refer to a composition in -which the protein or peptide forms the major component of the composition, such as constituting about 50% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “—fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purifications steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater—fold purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column. in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding .partners to an insoluble matrix. The column material is then able to specifically adsorb the substance from the solution. Elution occurs by changing the conditions to those in which binding will not occur (alter pH, ionic. strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

IV. Antibody Production

A. Generation of Monoclonal Antibodies

In another aspect, the present invention contemplates an antibody that is immunoreactive with NKG2D extracellular domains. An antibody can be a polyclonal or a monoclonal antibody. In a preferred embodiment, an antibody is a monoclonal antibody. Means for preparing and characterizing antibodies are well known in the art (see, e.g., Howell and Lane, 1988).

Briefly, a polyclonal antibody is prepared by immunizing an animal with an immunogen comprising a polypeptide of the present invention and collecting antisera from that immunized animal. A wide range of animal species can be used for the production of antisera. Typically an animal used for production of anti-antisera is a non-human animal including rabbits, mice, rats, hamsters, pigs or horses. Because of the relatively large blood volume of rabbits, a rabbit is a preferred choice for production of polyclonal antibodies.

Antibodies, both polyclonal and monoclonal, specific for isoforms of antigen may be prepared using. conventional immunization techniques, as will be generally known to those of skill in the art. A composition containing antigenic epitopes of the compounds of the present invention can be used to immunize one or more experimental animals, such as a rabbit or mouse, which will then proceed to produce specific antibodies against the compounds of the present invention. Polyclonal antisera may be obtained, after allowing time for antibody generation, simply by bleeding the animal and preparing serum samples from the whole blood.

Additionally, it is proposed that monoclonal antibodies specific to the particular NKG2D alleles may be utilized in other useful applications. For example, their use in immunoabsorbent protocols may be useful in purifying native or recombinant NKG2D isoforms or variants thereof.

In general, both poly- and monoclonal antibodies against NKG2D-related antigens may be used in a variety of embodiments. For example, they may be employed in antibody cloning protocols to obtain cDNAs or genes encoding NKG2D or fragments thereof. Means for preparing and characterizing antibodies are well known in the art (See, e.g., Harlow and Lane, 1988; incorporated herein by reference). More specific examples of monoclonal antibody preparation are give in the examples below.

As is well known in the art, a given composition may vary in its immunogenicity. It is often necessary therefore to boost the host immune system, as may be achieved by coupling a peptide or polypeptide immunogen to a carrier. Exemplary and preferred carriers are keyhole limpet hemocyanin (KLH) and bovine serum albumin (BSA). Other albumins such as ovalbumin, mouse serum albumin or rabbit serum albumin can also be used as carriers. Means for conjugating a polypeptide to a carrier protein are well known in the art and include glutaraldehyde, m-maleimidobencoyl-N-hydroxysuccinimide ester, carbodiimide and bis-biazotized benzidine.

As is also well known in the art, the immunogenicity of a particular immunogen composition can be enhanced by the use of non-specific stimulators of the immune response, known as adjuvants. Exemplary and preferred adjuvants include complete Freund's adjuvant (a non-specific stimulator of the immune response containing killed Mycobacterium tuberculosis), incomplete Freund's adjuvants and aluminum hydroxide adjuvant.

The amount of immunogen composition used in the production of polyclonal antibodies varies upon the nature of the immunogen as well as the animal used for immunization. A variety of routes can be used to administer the immunogen (subcutaneous, intramuscular, intradermal, intravenous and intraperitoneal). The production of polyclonal antibodies may be monitored by sampling blood of the immunized animal at various points following immunization. A second, booster, injection may also be given. The process of boosting and titering is repeated until a suitable titer is achieved. When a desired level of immunogenicity is obtained, the immunized animal can be bled and the serum isolated and stored, and/or the animal can be used to generate mAbs.

MAbs may be readily prepared through use of well-known techniques, such as those exemplified in U.S. Pat. No. 4,196,265, incorporated herein by reference. Typically, this technique involves immunizing a suitable animal with a selected immunogen composition, e.g., a purified or partially purified NKG2D protein, polypeptide or peptide or cell expressing high levels of NKG2D. The immunizing composition is administered in a manner effective to stimulate antibody producing cells. Rodents such as mice and rats are preferred animals, however, the use of rabbit, sheep frog cells is also possible. The use of rats may provide certain advantages (Goding, 1986), but mice are preferred, with the BALB/c mouse being most preferred as this is most routinely used and generally gives a higher percentage of stable fusions.

Following immunization, somatic cells with the potential for producing antibodies, specifically B-lymphocytes (B-cells), are selected for use in the mAb generating protocol. These cells may be obtained from biopsied spleens, tonsils or lymph nodes, or from a peripheral blood sample. Spleen cells and peripheral blood cells are preferred, the former because they are a rich source of antibody-producing cells that are in the dividing plasmablast stage, and the latter because peripheral blood is easily accessible. Often, a panel of animals will have been immunized and the spleen of animal with the highest antibody titer will be removed and the spleen lymphocytes obtained by homogenizing the spleen with a syringe. Typically, a spleen from an immunized mouse contains approximately 5×10⁷ to 2×10⁸ lymphocytes.

The antibody-producing B lymphocytes from the immunized animal are then fused with cells of an immortal myeloma cell, generally one of the same species as the animal that was immunized. Myeloma cell lines suited for use in hybridoma-producing fusion procedures preferably are non-antibody-producing, have high fusion efficiency, and enzyme deficiencies that render then incapable of growing in certain selective media which support the growth of only the desired fused cells (hybridomas).

Any one of a number of myeloma cells may be used, as are known to those of skill in the art (Goding, 1986; Campbell, 1984). For example, where the immunized animal is a mouse, one may use P3-X63/Ag8, P3-X63-Ag8.653, NS1/1.Ag 4 1, Sp210-Ag14, FO, NSO/U, MPC-11, MPC11-X45-GTG 1.7 and S194/5XX0 Bul; for rats, one may use R210.RCY3, Y3-Ag 1.2.3, IR983F and 4B210; and U-266, GM1500-GRG2, LICR-LON-HMy2 and UC729-6 are all useful in connection with cell fusions.

Methods for generating hybrids of antibody-producing spleen or lymph node cells and myeloma cells usually comprise mixing somatic cells with myeloma cells in a 2:1 ratio, though the ratio may vary from about 20:1 to about 1:1, respectively, in the presence of an agent or agents (chemical or electrical) that promote the fusion of cell membranes. Fusion methods using Sendai virus have been described (Kohler and Milstein, 1975; 1976), and those using polyethylene glycol (PEG), such as 37% (v/v) PEG, by Gefter et al., (1977). The use of electrically induced fusion methods is also appropriate (Goding, 1986).

Fusion procedures usually produce viable hybrids at low frequencies, around 1×10⁻⁶ to 1×10⁻⁸. However, this does not pose a problem, as the viable, fused hybrids are differentiated from the parental, unfused cells (particularly the unfused myeloma cells that would normally continue to divide indefinitely) by culturing in a selective medium. The selective medium is generally one that contains an agent that blocks the de novo synthesis of nucleotides in the tissue culture media. Exemplary and preferred agents are aminopterin, methotrexate, and azaserine. Aminopterin and methotrexate block de novo synthesis of both purines and pyrimidines, whereas azaserine blocks only purine synthesis. Where aminopterin or methotrexate is used, the media is supplemented with hypoxanthine and thymidine as a source of nucleotides (HAT medium). Where azaserine is used, the media is supplemented with hypoxanthine.

The preferred selection medium is HAT. Only cells capable of operating nucleotide salvage pathways are able to survive in HAT medium. The myeloma cells are defective in key enzymes of the salvage pathway, e.g., hypoxanthine phosphoribosyl transferase (HPRT), and they cannot survive. The B-cells can operate this pathway, but they have a limited life span in culture and generally die within about two weeks. Therefore, the only cells that can survive in the selective media are those hybrids formed from myeloma and B-cells.

This culturing provides a population of hybridomas from which specific hybridomas are selected. Typically, selection of hybridomas is performed by culturing the cells by single-clone dilution in microtiter plates, followed by testing the individual clonal supernatants (after about two to three weeks) for the desired reactivity. The assay should be sensitive, simple and rapid, such as radioimmunoassays, enzyme immunoassays, cytotoxicity assays, plaque assays, dot immunobinding assays, and the like.

The selected hybridomas would then be serially diluted and cloned into individual antibody-producing cell lines, which clones can then be propagated indefinitely to provide mAbs. The cell lines may be exploited for mAb production in two basic ways. A sample of the hybridoma can be injected (often into the peritoneal cavity) into a histocompatible animal of the type that was used to provide the somatic and myeloma cells for the original fusion. The injected animal develops tumors secreting the specific monoclonal antibody produced by the fused cell hybrid. The body fluids of the animal, such as serum or ascites fluid, can then be tapped to provide mAbs in high concentration. The individual cell lines could also be cultured in vitro, where the mAbs are naturally secreted into the culture medium from which they can be readily obtained in high concentrations. mAbs produced by either means may be further purified, if desired, using filtration, centrifugation and various chromatographic methods such as HPLC or affinity chromatography.

V. Cells

A. NKG2D Expressing Cells

The present invention, in one embodiment, will employ cells that naturally express NKG2D. Such cells include most γδ T-cells, CD8⁺ αβ T cells and NK cells. In other contexts, cells may be engineered to express NKG2D, or a suitable derivative thereof. General attributes of cells suitable for such engineering include any antigen-specific or regulatory T-cells (CD8 or CD4 αβ T-cells) that are expanded in vitro, transduced for enhanced or de novo expression of NKG2D or a suitable fusion protein and infused into patients for treatment of tumors or viral or other microbial diseases.

B. Expression Constructs

The term “expression vector” or “expression construct” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found Vectors include plasmids, cosmids, viruses (bacteriophage, animal viruses, and plant viruses), and artificial chromosomes (e.g., YACs). One of skill in the art would be well equipped to construct a vector through standard recombinant techniques (see, for example, Maniatis et al., 1988 and Ausubel et al., 1994, both incorporated herein by reference).

These terms refer to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in -a particular host cell. In addition to control sequences that govern transcription and translation, vectors and expression vectors may contain nucleic acid sequences that serve other functions as well and are described infra.

i) Promoters and Enhancers

A “promoter” is a control sequence that is a region of a nucleic acid sequence at which initiation and rate of transcription are controlled. It may contain genetic elements at which regulatory proteins and molecules may bind, such as RNA polymerase and other transcription factors, to initiate the specific transcription a nucleic acid sequence. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence.

A promoter generally comprises a sequence that functions to position the start site for RNA synthesis. The best known example of this is the TATA box, but in some promoters lacking a TATA box, such as, for example, the promoter for the mammalian terminal deoxynucleotidyl transferase gene and the promoter for the SV40 late genes, a discrete element overlying the start site itself helps to fix the place of initiation. Additional promoter elements regulate the frequency of transcriptional initiation. Typically, these are located in the region 30-110 bp upstream of the start site, although a number of promoters have been shown to contain functional elements downstream of the start site as well. To bring a coding sequence “under the control of” a promoter, one positions the 5′ end of the transcription initiation site of the transcriptional reading frame “downstream” of (i.e., 3′ of) the chosen promoter. The “upstream” promoter stimulates transcription of the DNA and promotes expression of the encoded RNA.

The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either cooperatively or independently to activate transcription. A promoter may or may not be used in conjunction with an “enhancer,” which refers to a cis-acting regulatory sequence involved in the transcriptional activation of a nucleic acid sequence.

A promoter may be one naturally associated with a nucleic acid sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other virus, or prokaryotic or eukaryotic cell, and promoters or enhancers not “naturally occurring,” i.e., containing different elements of different transcriptional regulatory regions, and/or mutations that alter expression. For example, promoters that are most commonly used in recombinant DNA construction include the β-lactamase (penicillinase), lactose and tryptophan (trp) promoter systems. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. Nos. 4,683,202 and 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the organelle, cell type, tissue, organ, or organism chosen for expression. Those of skill in the art of molecular biology generally know the use of promoters, enhancers, and cell type combinations for protein expression, (see, for example Sambrook et al. 1989, incorporated herein by reference). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Additionally any promoter/enhancer combination (as per, for example, the Eukaryotic Promoter Data Base EPDB, http://www.epd.isb-sib.ch/) could also be used to drive expression. Use of a T3, T7 or SP6 cytoplasmic expression system is another possible embodiment. Eukaryotic cells can support cytoplasmic transcription from certain bacterial promoters if the appropriate bacterial polymerase is provided, either as part of the delivery complex or as an additional genetic expression construct.

Table 2 lists non-limiting examples of elements/promoters that may be employed, in the context of the present invention, to regulate the expression of a RNA. Table 3 provides non-limiting examples of inducible elements, which are regions of a nucleic acid sequence that can be activated in response to a specific stimulus.

TABLE 2
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton |
| | et al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn |
| | et al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-Dra | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Ornitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tronche et al., 1989, 1990 |
| α-Fetoprotein | Godbout et al., 1988; Campere et al., 1989 |
| γ-Globin | Bodine et al., 1987; Perez-Stable et al., 1990 |
| β-Globin | Trudel et al., 1987 |
| c-fos | Cohen et al., 1987 |
| c-HA-ras | Triesman, 1986; Deschamps et al., 1985 |
| Insulin | Edlund et al., 1985 |
| Neural Cell Adhesion Molecule | Hirsch et al., 1990 |
| (NCAM) | |
| α₁-Antitrypsin | Latimer et al., 1990 |
| H2B (TH2B) Histone | Hwang et al., 1990 |
| Mouse and/or Type I Collagen | Ripe et al., 1989 |
| Glucose-Regulated Proteins | Chang et al., 1989 |
| (GRP94 and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek |
| | et al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake |
| | et al., 1988; Campbell and/or Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Choi et al., 1988; Reisman et al., |
| | 1989 |
| Papilloma Virus | Campo et al., 1983; Lusky et al., 1983; Spandidos and/or |
| | Wilkie, 1983; Spalholz et al., 1985; Lusky et al., 1986; |
| | Cripe et al., 1987; Gloss et al., 1987; Hirochika et al., |
| | 1987; Stephens et al., 1987 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits |
| | et al., 1988; Feng et al., 1988; Takebe et al., 1988; Rosen |
| | et al., 1988; Berkhout et al., 1989; Laspia et al., 1989; |
| | Sharp et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 3
Inducible Elements

| Element | Inducer | References |
|---|---|---|
| MT II | Phorbol Ester (TFA) | Palmiter et al., 1982; Haslinger |
| | Heavy metals | et al., 1985; Searle et al., 1985; |
| | | Stuart et al., 1985; Imagawa |
| | | et al., 1987, Karin et al., 1987; |
| | | Angel et al., 1987b; McNeall |
| | | et al., 1989 |
| MMTV (mouse mammary | Glucocorticoids | Huang et al., 1981; Lee et al., |
| tumor virus) | | 1981; Majors et al., 1983; |
| | | Chandler et al., 1983; Lee et al., |
| | | 1984; Ponta et al., 1985; Sakai |
| | | et al., 1988 |
| β-Interferon | Poly(rI)x | Tavernier et al., 1983 |
| | Poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle | Hug et al., 1988 |
| | Disease Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a, 1990b |
| | Antigen | |
| Proliferin | Phorbol Ester-TPA | Mordacq et al., 1989 |
| Tumor Necrosis Factor α | PMA | Hensel et al., 1989 |
| Thyroid Stimulating | Thyroid Hormone | Chatterjee et al., 1989 |
| Hormone α Gene | | |

The identity of tissue-specific promoters or elements, as well as assays to characterize their activity, is well known to those of skill in the art. Nonlimiting examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), and human platelet endothelial cell adhesion molecule-1 (Almendro et al., 1996).

ii) Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picomavirus family (polio and encephalomyocarditis) have been described (Pelletier and Sonenberg, 1988), as well an IRES from a mammalian message (Macejak and Samow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. Nos. 5,925,565 and 5,935,819, each herein incorporated by reference).

iii) Multiple Cloning Sites

Vectors can include a multiple cloning site (MCS), which is a nucleic acid region that contains multiple restriction enzyme sites, any of which can be used in conjunction with standard recombinant technology to digest the vector (see, for example, Carbonelli et al. 1999; Levenson et al. 1998; and Cocea 1997, incorporated herein by reference). “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector. “Ligation” refers to the process of forming phosphodiester bonds between two nucleic acid fragments, which may or may not be contiguous with each other. Techniques involving restriction enzymes and ligation reactions are well known to those of skill in the art of recombinant technology.

iv) Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression (see, for example, Chandler et al., 1997, herein incorporated by reference).

v) Termination Signals

The vectors or constructs of the present invention will generally comprise at least one termination signal. A “termination signal” or “terminator” is comprised of the DNA sequences involved in specific termination of an RNA transcript by an RNA polymerase. Thus, in certain embodiments a termination signal that ends the production of an RNA transcript is contemplated. A terminator may be necessary in vivo to achieve desirable message levels.

In eukaryotic systems, the terminator region may also comprise specific DNA sequences that permit site-specific cleavage of the new transcript so as to expose a polyadenylation site. This signals a specialized endogenous polymerase to add a stretch of about 200 A residues (polyA) to the 3′ end of the transcript. RNA molecules modified with this polyA tail appear to more stable and are translated more efficiently. Thus, in other embodiments involving eukaryotes, it is preferred that that terminator comprises a signal for the cleavage of the RNA, and it is more preferred that the terminator signal promotes polyadenylation of the message. The terminator and/or polyadenylation site elements can serve to enhance message levels and to minimize read through from the cassette into other sequences.

Terminators contemplated for use in the invention include any known terminator of transcription described herein or known to one of ordinary skill in the art, including but not limited to, for example, the termination sequences of genes, such as for example the bovine growth hormone terminator or viral termination sequences, such as for example the SV40 terminator. In certain embodiments, the termination signal may be a lack of transcribable or translatable sequence, such as due to a sequence truncation.

vi) Polyadenylation Signals

In expression, particularly eukaryotic expression, one will typically include a polyadenylation signal to effect proper polyadenylation of the transcript. The nature of the polyadenylation signal is not believed to be crucial to the successful practice of the invention, and any such sequence may be employed. Preferred embodiments include the SV40 polyadenylation signal or the bovine growth hormone polyadenylation signal, convenient and known to function well in various target cells. Polyadenylation may increase the stability of the transcript or may facilitate cytoplasmic transport.

vii) Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

viii) Selectable and Screenable Markers

In certain embodiments of the invention, cells containing a nucleic acid construct of the present invention may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

ix) Plasmid Vectors

In certain embodiments, a plasmid vector is contemplated for use to transform a host cell. In general, plasmid vectors containing replicon and control sequences which are derived from species compatible with the host cell are used in connection with these hosts. The vector ordinarily carries a replication site, as well as marking sequences which are capable of providing phenotypic selection in transformed cells. In a non-limiting example, E. coli is often transformed using derivatives of pBR322, a plasmid derived from an E. coli species. pBR322 contains genes for ampicillin and tetracycline resistance and thus provides easy means for identifying transformed cells. The pBR plasmid, or other microbial plasmid or phage must also contain, or be modified to contain, for example, promoters which can be used by the microbial organism for expression of its own proteins.

In addition, phage vectors containing replicon and control sequences that are compatible with the host microorganism can be used as transforming vectors in connection with these hosts. For example, the phage lambda GEM™-11 may be utilized in making a recombinant phage vector which can be used to transform host cells, such as, for example, E. coli LE392.

Further useful plasmid vectors include pIN vectors (Inouye et al., 1985); and pGEX vectors, for use in generating glutathione S-transferase (GST) soluble fusion proteins for later purification and separation or cleavage. Other suitable fusion proteins are those with β-galactosidase, ubiquitin, and the like.

Bacterial host cells, for example, E. coli, comprising the expression vector, are grown in any of a number of suitable media, for example, LB. The expression of the recombinant protein in certain vectors may be induced, as would be understood by those of skill in the art, by contacting a host cell with an agent specific for certain promoters, e.g., by adding IPTG to the media or by switching incubation to a higher temperature. After culturing the bacteria for a further period, generally of between 2 and 24 h, the cells are collected by centrifugation and washed to remove residual media.

x) Viral Vectors

The ability of certain viruses to infect cells or enter cells via receptor-mediated endocytosis, and to integrate into host cell genome and express viral genes stably and efficiently have made them attractive candidates for the transfer of foreign nucleic acids into cells. Non-limiting examples of virus vectors that may be used to deliver a nucleic acid of the present invention are described below.

I. Adenoviral Vectors

A particular method for delivery of the nucleic acid involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and (b) to ultimately express a tissue or cell-specific construct that has been cloned therein. Knowledge of the genetic organization or adenovirus, a 36 kb, linear, double-stranded DNA virus, allows substitution of large pieces of adenoviral DNA with foreign sequences up to 7 kb (Grunhaus and Horwitz 1992).

2. AAV Vectors

The nucleic acid may be introduced into the cell using adenovirus assisted transfection. Increased transfection efficiencies have been reported in cell systems using adenovirus coupled systems (Kelleher and Vos 1994; Cotten et al. 1992; Curiel 1994). Adeno-associated virus (AAV) is an attractive vector system for use according to the present invention as it has a high frequency of integration and it can infect nondividing cells, thus making it useful for delivery of genes into mammalian cells, for example, in tissue culture (Muzyczka 1992) or in vivo. AAV has a broad host range for infectivity (Tratschin et al. 1984; Laughlin et al. 1986; Lebkowski et al. 1988; McLaughlin et al. 1988). Details concerning the generation and use of rAAV vectors are described in U.S. Pat. Nos. 5,139,941 and 4,797,368, each incorporated herein by reference.

3. Retroviral Vectors

Retroviruses integrate their genes into the host genome have the advantage of transferring a large amount of foreign genetic material, infecting a broad spectrum of species and cell types, and of being packaged in special cell-lines (Miller, 1992).

In order to construct a retroviral vector, a nucleic acid of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into a special cell line (e.g., by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

Lentiviruses are complex retroviruses, which, in addition to the common retroviral genes gag, pol, and env, contain other genes with regulatory or structural function. Lentiviral vectors are well known in the art (see, for example, Naldini et al., 1996; Zufferey et al., 1997; Blomer et al., 1997; U.S. Pat. Nos. 6,013,516 and 5,994,136). Some examples of lentivirus include the Human Immunodeficiency Viruses: HIV-1, HIV-2 and the Simian Immunodeficiency Virus: SIV. Lentiviral vectors have been generated by multiply attenuating the HIV virulence genes, for example, the genes env, vif, vpr, vpu and nef are deleted making the vector biologically safe.

Recombinant lentiviral vectors are capable of infecting non-dividing cells and can be used for both in vivo and ex vivo gene transfer and expression of nucleic acid sequences. For example, recombinant lentivirus capable of infecting a non-dividing cell wherein a suitable host cell is transfected with two or more vectors carrying the packaging functions, namely gag, pol and env, as well as rev and tat is described in U.S. Pat. No. 5,994,136, incorporated herein by reference. One may target the recombinant virus by linkage of the envelope protein with an antibody or a particular ligand for targeting to a receptor of a particular cell-type. By inserting a sequence (including a regulatory region) of interest into the viral vector, along with another gene which encodes the ligand for a receptor on a specific target cell, for example, the vector is now target-specific.

4. Other Viral Vectors

Other viral vectors may be employed as delivery constructs in the present invention. Vectors derived from viruses such as vaccinia virus (Ridgeway, 1988; Baichwal and Sugden, 1986; Coupar et al., 1988), sindbis virus, cytomegalovirus and herpes simplex virus may be employed. They offer several attractive features for various mammalian cells (Friedmann, 1989; Ridgeway, 1988; Baichwal and Sugden, 1986; Coupar et al., 1988; Horwich et al., 1990).

5. Delivery Using Modified Viruses

A nucleic acid to be delivered may be housed within an infective virus that has been engineered to express a specific binding ligand. The virus particle will thus bind specifically to the cognate receptors of the target cell and deliver the contents to the cell. A novel approach designed to allow specific targeting of retrovirus vectors was developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

C. Methods for Transforming Host Cells

There are a number of ways in which nucleic acids may introduced into cells. Viral methods rely on the use of viral vectors listed above. A variety of non-viral transduction methods, are outlined below.

Suitable methods for nucleic acid delivery for transformation of an organelle, a cell, a tissue or an organism for use with the current invention are believed to include virtually any method by which a nucleic acid (e.g., DNA) can be introduced into an organelle, a cell, a tissue or an organism, as described herein or as would be known to one of ordinary skill in the art. Such methods include, but are not limited to, direct delivery of DNA such as by ex vivo transfection (Wilson et al., 1989, Nabel et al., 1989), by injection (U.S. Pat. Nos. 5,994,624, 5,981,274, 5,945,100, 5,780,448, 5,736,524, 5,702,932, 5,656,610, 5,589,466 and 5,580,859, each incorporated herein by reference), including microinjection (Harlan and Weintraub, 1985; U.S. Pat. No. 5,789,215, incorporated herein by reference); by electroporation (U.S. Pat. No. 5,384,253, incorporated herein by reference; Tur-Kaspa et al. 1986; Potter et al., 1984); by calcium phosphate precipitation (Graham and Van Der Eb 1973; Chen and Okayama, 1987; Rippe et al., 1990); by using DEAE-dextran followed by polyethylene glycol (Gopal, 1985); by direct sonic loading (Fechheimer et al. 1987); by liposome mediated transfection (Nicolau and Sene, 1982; Fraley et al., 1979; Nicolau et al., 1987; Wong et al., 1980; Kaneda et al., 1989; Kato et al., 1991) and receptor-mediated transfection (Wu and Wu, 1987; Wu and Wu, 1988); by microprojectile bombardment (PCT Application Nos. WO 94/09699 and 95/06128; U.S. Pat. Nos. 5,610,042; 5,322,783 5,563,055, 5,550,318, 5,538,877 and 5,538,880, and each incorporated herein by reference); by agitation with silicon carbide fibers (Kaeppler et al., 1990; U.S. Pat. Nos. 5,302,523 and 5,464,765, each incorporated herein by reference); by Agrobacterium-mediated transformation (U.S. Pat. Nos. 5,591,616 and 5,563,055, each incorporated herein by reference); by PEG-mediated transformation of protoplasts (Omirulleh et al. 1993; U.S. Pat. Nos. 4,684,611 and 4,952,500, each incorporated herein by reference); by desiccation/inhibition-mediated DNA uptake (Potrykus et al., 1985), and any combination of such methods. Through the application of techniques such as these, organelle(s), cell(s), tissue(s) or organism(s) may be stably or transiently transformed.

i) Ex Vivo Transformation

Methods for tranfecting vascular cells and tissues removed from an organism in an ex vivo setting are known to those of skill in the art. For example, cannine endothelial cells have been genetically altered by retrovial gene transfer in vitro and transplanted into a canine (Wilson et al., 1989). In another example, yucatan minipig endothelial cells were transfected by retrovirus in vitro and transplanted into an artery using a double-balloonw catheter (Nabel et al., 1989). Thus, it is contemplated that cells or tissues may be removed and tranfected ex vivo using the nucleic acids of the present invention. In particular aspects, the transplanted cells or tissues may be placed into an organism. In preferred facets, a nucleic acid is expressed in the transplated cells or tissues.

ii) Injection

In certain embodiments, a nucleic acid may be delivered to an organelle, a cell, a tissue or an organism via one or more injections (i.e., a needle injection), such as, for example, subcutaneously, intradermally, intramuscularly, intervenously, intraperitoneally, etc. Methods of injection of vaccines are well known to those of ordinary skill in the art (e.g., injection of a composition comprising a saline solution). Further embodiments of the present invention include the introduction of a nucleic acid by direct microinjection. Direct microinjection has been used to introduce nucleic acid constructs into Xenopus oocytes (Harland and Weintraub 1985). The amount of DNA used may vary upon the nature of the antigen as well as the organelle, cell, tissue or organism used.

iii) Electroporation

In certain embodiments of the present invention, a nucleic acid is introduced into an organelle, a cell, a tissue or an organism via electroporation. Electroporation involves the exposure of a suspension of cells and DNA to a high-voltage electric discharge. In some variants of this method, certain cell wall-degrading enzymes, such as pectin-degrading enzymes, are employed to render the target recipient. cells more susceptible to transformation by electroporation than untreated cells (U.S. Pat. No. 5,384,253, incorporated herein by reference). Alternatively, recipient cells can be made more susceptible to transformation by mechanical wounding.

Transfection of eukaryotic cells using electroporation has been quite successful. Mouse pre-B lymphocytes have been transfected with human kappa-immunoglobulin genes (Potter et al. 1984), and rat hepatocytes have been transfected with the chloramphenicol acetyltransferase gene (Tur-Kaspa et al. 1986) in this manner.

iv) Calcium Phosphate

In other embodiments of the present invention, a nucleic acid is introduced to the cells using calcium phosphate precipitation. Human KB cells have been transfected with adenovirus 5 DNA (Graham and Van Der Eb 1973) using this technique. Also in this manner, mouse L(A9), mouse C127, CHO, CV-1, BHK, NIH3T3 and HeLa cells were transfected with a neomycin marker gene (Chen and Okayama 1987), and rat hepatocytes were transfected with a variety of marker genes (Rippe et al. 1990).

v) DEAE-Dextran

In another embodiment, a nucleic acid is delivered into a cell using DEAE-dextran followed by polyethylene glycol. In this manner, reporter plasmids were introduced into mouse myeloma and erythroleukemia cells (Gopal 1985).

vi) Sonication Loading

Additional embodiments of the present invention include the introduction of a nucleic acid by direct sonic loading. LTK- fibroblasts have been transfected with the thymidine kinase gene by sonication loading (Fechheimer et al. 1987).

vii) Liposome-Mediated Transfection

In a further embodiment of the invention, a nucleic acid may be entrapped in a lipid complex such as, for example, a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat 1991). Also contemplated is an nucleic acid complexed with Lipofectamine (Gibco BRL) or Superfect (Qiagen).

Liposome-mediated nucleic acid delivery and expression of foreign DNA in vitro has been very successful (Nicolau and Sene 1982; Fraley et al. 1979; Nicolau et al. 1987). The feasibility of liposome-mediated delivery and expression of foreign DNA in cultured chick embryo, HeLa and hepatoma cells has also been demonstrated (Wong et al. 1980).

In certain embodiments of the invention, a liposome may be complexed with a hemagglutinating virus (HVJ). This has been shown to facilitate fusion with the cell membrane and promote cell entry of liposome-encapsulated DNA (Kaneda et al. 1989). In other embodiments, a liposome may be complexed or employed in conjunction with nuclear non-histone chromosomal proteins (HMG-1) (Kato et al. 1991). In yet further embodiments, a liposome may be complexed or employed in conjunction with both HVJ and HMG-1. In other embodiments, a delivery vehicle may comprise a ligand and a liposome.

viii) Receptor Mediated Transfection

Still further, a nucleic acid may be delivered to a target cell via receptor-mediated delivery vehicles. These take advantage of the selective uptake of macromolecules by receptor-mediated endocytosis that will be occurring in a target cell. In view of the cell type-specific distribution of various receptors, this delivery method adds another degree of specificity to the present invention.

Certain receptor-mediated gene targeting vehicles comprise a cell receptor-specific ligand and a nucleic acid-binding agent. Others comprise a cell receptor-specific ligand to which the nucleic acid to be delivered has been operatively attached. Several ligands have been used for receptor-mediated gene transfer (Wu and Wu, 1987; Wagner et al., 1990; Perales et al., 1994; Myers, EPO 0273085), which establishes the operability of the technique. Specific delivery in the context of another mammalian cell type has been described (Wu and Wu, 1993; incorporated herein by reference). In certain aspects of the present invention, a ligand will be chosen to correspond to a receptor specifically expressed on the target cell population.

In other embodiments, a nucleic acid delivery vehicle component of a cell-specific nucleic acid targeting vehicle may comprise a specific binding ligand in combination with a liposome. The nucleic acid(s) to be delivered are housed within the liposome and the specific binding ligand is functionally incorporated into the liposome membrane. The liposome will thus specifically bind to the receptor(s) of a target cell and deliver the contents to a cell. Such systems have been shown to be functional using systems in which, for example, epidermal growth factor (EGF) is used in the receptor-mediated delivery of a nucleic acid to cells that exhibit upregulation of the EGF receptor.

In still further embodiments, the nucleic acid delivery vehicle component of a targeted delivery vehicle may be a liposome itself, which will preferably comprise one or more lipids or glycoproteins that direct cell-specific binding. For example, lactosyl-ceramide, a galactose-terminal asialganglioside, have been incorporated into liposomes and observed an increase in the uptake of the insulin gene by hepatocytes (Nicolau et al. 1987). It is contemplated that the tissue-specific transforming constructs of the present invention can be specifically delivered into a target cell in a similar manner.

ix) Microprojectile Bombardment

Microprojectile bombardment techniques can be used to introduce a nucleic acid into at least one, organelle, cell, tissue or organism (U.S. Pat. Nos. 5,550,318; 5,538,880; 5,610,042; and PCT Application WO 94/09699; each of which is incorporated herein by reference). This method depends on the ability to accelerate DNA-coated microprojectiles to a high velocity allowing them to pierce cell membranes and enter cells without killing them (Klein et al., 1987). There are a wide variety of microprojectile bombardment techniques known in the art, many of which are applicable to the invention.

In microprojectile bombardment, one or more particles may be coated with at least one nucleic acid and delivered into cells by a propelling force. Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold particles or beads. Exemplary particles include those comprised of tungsten, platinum, and preferably, gold. It is contemplated that in some instances DNA precipitation onto metal particles would not be necessary for DNA delivery to a recipient cell using microprojectile bombardment. However, it is contemplated that particles may contain DNA rather than be coated with DNA. DNA-coated particles may increase the level of DNA delivery via particle bombardment but are not, in and of themselves, necessary.

For the bombardment, cells in suspension are concentrated on filters or solid culture medium. Alternatively, immature embryos or other target cells may be arranged on solid culture medium. The cells to be bombarded are positioned at an appropriate distance below the macroprojectile stopping plate.

An illustrative embodiment of a method for delivering DNA into a cell (e.g., a plant cell) by acceleration is the Biolistics Particle Delivery System, which can be used to propel particles coated with DNA or cells through a screen, such as a stainless steel or Nytex screen, onto a filter surface covered with cells, such as for example, a monocot plant cells cultured in suspension. The screen disperses the particles so that they are not delivered to the recipient cells in large aggregates. It is believed that a screen intervening between the projectile apparatus and the cells to be bombarded reduces the size of projectiles aggregate and may contribute to a higher frequency of transformation by reducing the damage inflicted on the recipient cells by projectiles that are too large.

VI. Treatment of Various Disease States

In accordance with the present invention, applicants propose the use of NKG2D ligands or derivatives thereof to stimulate NKG2D expressing T-cells. In particular, applicants envision the use of such ligands to stimulate immune responses in a variety of clinical situations.

A. Obtaining T-Cell Populations

Antigen-specific T-cells can be directly isolated from peripheral blood or tissue from patients using, for example, HLA-peptide complex tetramer technology (Altman et al., 1996) and in vitro expanded using established culture conditions in the presence of irradiated antigen-presenting cells, solid-phase anti-NKG2D and cytokines. Additional methods may include FACS sorting and/or techniques based on magnetic beads coupled with antibodies to enrich desired T-cell populations (Groh et al. 1998). Large numbers of such T-cell populations with demonstrated antigen-specificity can subsequently be infused into patients. Another disease treatment platform is envisioned by using derivatives of anti-NKG2D antibody, such as bi-specific antibodies, or of suitably engeneered ligands, to directly target T-cells systemically or locally in the body, with the goal to enhance their ability to execute effector functions (cytotoxicity and cytokine release) and to induce limited proliferation.

B. Treatment of Cancer

In accordance with one embodiment of the present invention, there is provided a method for treating various cancers, including breast cancer, lung cancer, prostate cancer, cervical cancer, testicular cancer, brain cancer, renal cancer, liver cancer, stomach cancer, colon cancer, pancreatic cancer, head & neck cancer, skin cancer and ovarian cancer. As discussed above, appropriate cell populations are stimulated using NKG2D ligands as described elsewhere in this document. Such populations may be stimulated in vivo by administration of ligands as part of a suitable pharmaceutical preparation. Alternatively, an appropriate cell population may be isolated from the cancer patient, stimulated ex vivo, and then reinfised into the patient. The infusion of stimulated cells may be intratumoral, into the tumoral vasculature, regional to the tumor, or systemically via intravenous or intraarterial infusion. Systemic administration is particularly advantageous when attempting to prevent or treat metastatic tumors.

C. Treatment of Viral Infection

In another embodiment, the present invention provides for treatment or prevention of viral infection. Viruses contemplated as treatable using methods of the present invention include cytomegalovirus, berpesvirus, human immunodeficiency virus, influenza virus and any others. Treatment is envisioned as described above, by infusion of ex vivo expanded T-cells derived from a patient or by in vivo targetting of specific T-cells using suitable derivatives of anti-NKG2D antibody or ligands. This method may be of particular use with patients who are partially immunocompromised as a result of therapeutic treatment (radiation, chemotherapy, cytostatica) or disease (AIDS), by providing mobilization of compromised T-cell function.

D. Stimulation of Cytokine Production

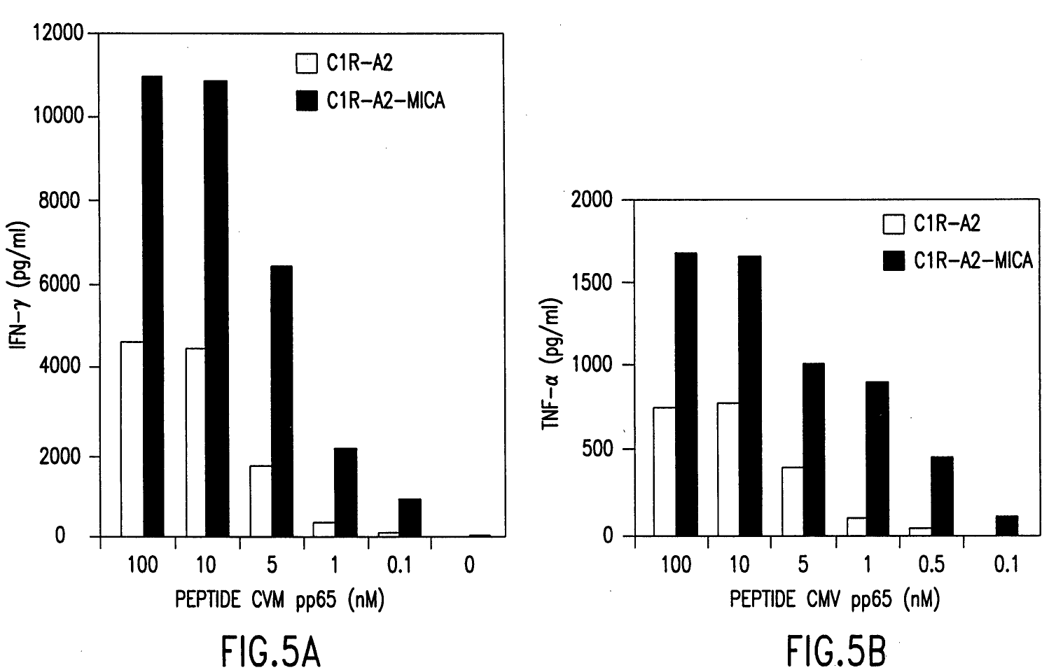

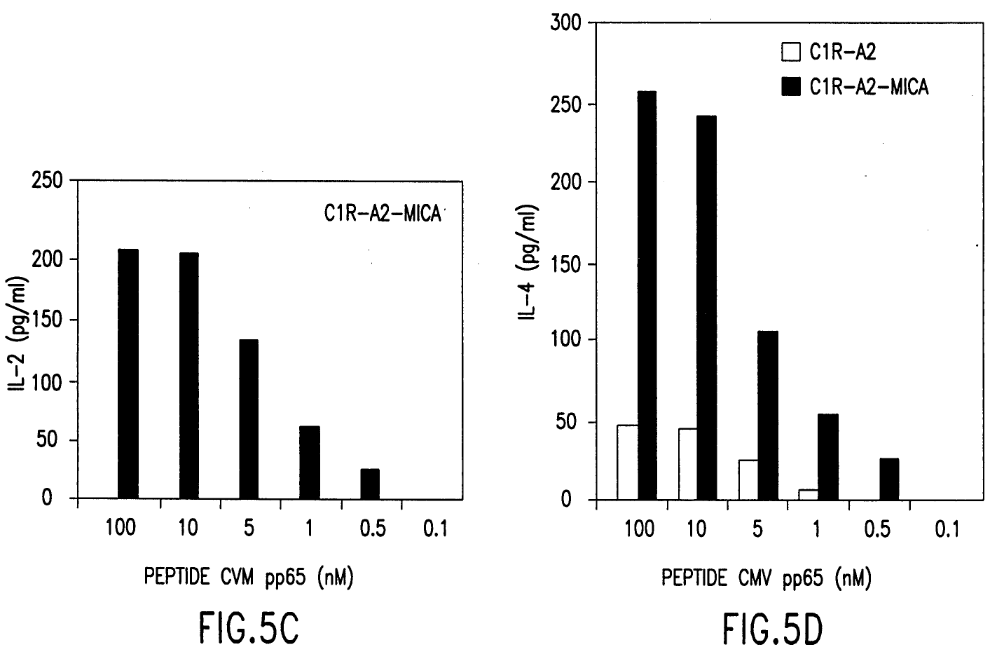

In yet another embodiment, the present invention provides for methods of stimulating the scretion of cytokines by lymphocytes. These cytokines include interferon-γ (IFN-γ), tumor necrosis factor-α (TNF-α), IL-2, IL-4 and GM-CSF, among others (Groh et al. 1998, 1999; see FIGS. 5-7). The stimulation of lymphokine production by anti-NKG2D antibody or a ligand derivative facilitates the proliferation of specific T-cell populations in vitro and may enhance their effector functions in vivo.

VII. Screening for Ligands of NKG2D

Within certain embodiments of the invention, methods are provided for screening for compounds that bind to, and hence activate, NKG2D. Within one example, a screening assay is performed in which cells expressing NKG2D are exposed to a test substance under suitable conditions and for a time sufficient to permit activation thereof. Activation may be measured, for example, by cellular proliferation, cytokine expression, or target cell lysis. Generally, the test substance is added in the form of a purified agent.

An alternative embodiment is a binding assay. Using an NKG2D receptor, one may measure binding to the receptor via a variety of methods, including alteration in electrophoretic mobility of the NKG2D (or fragment), competitive binding for NKG2D (as measured by loss of signal for labeled competitor), or any other suitable method. Also, industrial scale screenings of commercially available drug banks and peptide libraries for compounds binding to NKG2D are envisioned.

VIII. Kit Components

All the essential materials and reagents required for stimulating NKG2D, or fusion molecules thereof, may be assembled together in a kit. Such kits generally will comprise, in suitable means, distinct containers for each individual ligand. Such kits also may comprise, in suitable distinct containers, buffer for dilution of ligand. Other reagents may be growth factors or lymphokines/cytokines for culturing of stimulated cells.

IX. Pharmaceutical Compositions

For use according to the present application, it may be necessary to prepare pharmaceutical compositions—NKG2D ligands—in a form appropriate for the intended application. Generally, this will entail preparing compositions that are essentially free of pyrogens, as well as other impurities that could be harmful to cells of humans or animals.

One will generally desire to employ appropriate salts and buffers suitable for dilution of ligands. Buffers also will be employed when recombinant cells are introduced into a patient. Aqueous compositions of the present invention comprise an effective amount of the vector to cells, dissolved or dispersed in a pharmaceutically acceptable carrier or aqueous medium. Such compositions also are referred to as inocula. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes any and all solvents, dispersion media, coatings, antibacterial and antifungal agents, isotonic and absorption delaying agents and the like. The use of such media and agents for pharmaceutically-active substances is well known in the art. Except insofar as any conventional media or agent is incompatible with the vectors or cells of the present invention, its use in therapeutic compositions is contemplated. Supplementary active ingredients also can be incorporated into the compositions.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by intratumoral, intradermal, subcutaneous, intramuscular, intraperitoneal or intravenous injection. Such compositions would normally be administered as pharmaceutically acceptable compositions, described supra.

The active compounds may also be administered parenterally or intraperitoneally. Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various anti-bacterial an antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

For oral administration the polypeptides of the present invention may be incorporated with excipients and used in the form of non-ingestible mouthwashes and dentifrices. A mouthwash may be prepared incorporating the active ingredient in the required amount in an appropriate solvent, such as a sodium borate solution (Dobell's Solution). Alternatively, the active ingredient may be incorporated into an antiseptic wash containing sodium borate, glycerin and potassium bicarbonate. The active ingredient may also be dispersed in dentifrices, including: gels, pastes, powders and slurries. The active ingredient may be added in a therapeutically effective amount to a paste dentifrice that may include water, binders, abrasives, flavoring agents, foaming agents, and humectants.

The compositions of the present invention may be formulated in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

Upon formulation, solutions will be administered in a manner compatible with the dosage formulation and in such amount as is therapeutically effective. The formulations are easily administered in a variety of dosage forms such as injectable solutions, drug release capsules and the like.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

X. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Methods

CMV infection of fibroblasts and endothelial cells, antibodies and flow cytometry. Primary human fibroblast (HF) cultures were established from skin biopsies of healthy individuals and grown in Waymouths media (Gibco) supplemented with 10% fetal bovine serum (FBS; Hyclone) and standard concentrations of penicillin, streptomycin and glutamine. Human umbilical vein endothelial cells (HUVEC) were grown on fibronectin-coated plates (Upstate Technologies) in RPMI (Gibco), 20% FBS, HEPES (10 mM), non-essential amino acids (0.1 mM; Gibco), endothelial cell growth supplement (50 μg/ml; Becton Dickinson), sodium pyruvate (1 mM), glutamine (2 mM), and antibiotics (penicillin, streptomycin and fungizone). Early (1-5 passage) cells grown to confluency were infected with CMV strain AD169 [5 plaque-forming units (pfu)/cell; American Type Culture Collection (ATCC)] or strain VHL/e (2 pfu/cell) (Waldmann et al., 1989). Control infections were with UV-irradiated (10⁶ joules/100 μl virus stock) AD169, which produced positive immunostaining for CMV pp65 (mAb anti-CMV pp65; Virostat) but no staining for IE-1 (mAb NEA-9221; NEN Life Science Products), and with heat-inactivated AD169 and mock-infected cell lysate stock. HF and HUVEC were stained before and at various time points after infection or control or mock infection with mAb 6D4 (anti-MICA and MICB; Groh et al. 1998), mAb W6/32 (anti-pan MHC class I; Parham et al., 1979), or Ig isotype-matched control antibody (IgG2a) and examined by indirect immunofluorescence using phycoerythrin (PE)-conjugated goat F(ab′)₂ anti-mouse Ig (Biosource) and flow cytometry.

Immunohistochemistry of CMV-infected cell cultures and lung tissue. Cytospin preparations of infected and control HF were fixed in cold acetone, blocked with 20% normal goat and 20% human serum in Tris-buffered saline, and incubated with mAb NEA-9221 (anti-CMV IE-1), anti CMV pp65 mAb, or isotype control Ig. Bound antibody was stained with biotin-goat anti-mouse F(ab′)₂ Ig (Jackson ImmunoResearch Laboratories) and Streptavidin Alexa™ 594 conjugate (Molecular Probes). Infected and control HUVEC monolayers grown on glass chamber well slides (Nalge Nunc International Corp) were acetone-fixed, stained for MIC expression with mAb 6D4 as described above, blocked with Avidin/Biotin Blocking Kit (Vector Laboratories), and double-stained with mAb NEA-9221, biotin-goat anti-mouse F(ab′)₂ Ig and Streptavidin CY™ conjugate (Jackson ImmunoResearch Laboratories). Nuclei were stained with 4′-6 diamino-2-phenylindole (5 μg/ml; Sigma). Samples were examined using a Delta Vision system (Applied Precision). Cryostat sections of OCT compound-embedded and snap-frozen CMV interstitial pneumonia autopsy specimens, post-transplant for treatment of chronic myeloid leukemia (CML), were air-dried and acetone-fixed, and stained with mAb 6D4 and mAb CCH2 (anti-CMV delayed early DNA-binding protein p52; Dako) using the Envision Doublestain System (Dako) with the diamino benzidine and Fast Red peroxidase substrates as described by the manufacturer.

Generation and maintenance of the CMV pp65-specific T-cell clones and isolation of peripheral blood CD8⁺ αβ T-cells. The CD8 αβ T-cell clones (HLA-A2-restricted clones 88C7-470, 94C10-12, 19D1-66, 4H6-254, 59C11-292 and 2E9-269; HLA-A1-restricted clones 8E8-403, 21D9-306 and 30F4-297) were generated from short-term CMV-specific cytotoxic T-cell lines as previously described (McLaughlin-Taylor et al., 1994; Gilbert et al., 1996). In brief, peripheral blood mononuclear cells (PBMC) from CMV seropositive volunteers were stimulated with autologous fibroblasts infected with AD169 (at a multiplicity of infection of 5) at a ratio of 1:20 in RPMI media supplemented with 10% human serum, 2-mercaptoethanol (25 μM), glutamine, penicillin and streptomycin. Cultures were restimulated after 7 days with autologous CMV-infected fibroblasts, in the presence of autologous γ-irradiated PBMC and recombinant IL-2 (Proleukin-2, 5 U/ml; Chiron). After 7 additional days, CD4⁺ T-cells were depleted using CD4 Dynabeads (Dynal) and enriched CD8⁺ T-cells plated (0.5 cells per well) and grown as described above. CD8 αβ T-cell clones were tested for anti-CMV specificity in chromium release assays and further expanded in the presence of γ-irradiated PBMC, anti-CD3 (OKT3, 30 ng/ml; Orthobiotech) and IL-2 (50 U/ml). McLaughlin-Taylor et al. (1994); Gilbert et al. (1996).

CD28⁻/CD8 T-cells were isolated from unseparated peripheral blood from healthy donors by negative selection using the CD8 T-cell enrichment cocktail RosetteSep™ (StemCell Technologies) and by depletion of CD28 T-cells using magnetic Pan Mouse IgG Dynabeads (Dynal) precoated with anti-CD28 (mAb 9.3; Hara et al., 1985) on a magnetic particle concentrator (Dynal). By flow cytometry, the CD28⁻ CD8 T-cells were of at least 98% purity.

Cytotoxicity, cytokine release and T-cell proliferation assays. T-cell cytolytic activity was tested in standard 4-h ⁵¹ Cr-release assays with labeled targets cells that included HF (typed for HLA-A1 or -A2) that were infected with CMV AD169 or mock-infected, and transfectants of the B-lympboblastoid C1R cell line expressing HLA-1 or -A2 alone or together with MICA (Groh et al., 1998). Before exposure to the HLA-A1- or -A2-restricted CMV pp65-specific T-cell clones, the transfectants were pulsed with the specific naturally processed pp65 9-mer peptides YSEHPTFTS and NLVPMVATV, respectively (Wills et al., 1996), at the concentrations indicated in the figure legends. For blocking experiments, effector or target cells were incubated with saturating amounts of mAb 1D11 (anti-NKG2D), W6/32 (anti-pan HLA) or mAb 6D4 (anti-MIC), either alone or in combination, or with control Ig, for 30 min before exposure to T-cells. Assays were performed in triplicate and results scored in percent specific lysis according to the standard formula.

In the cytokine release assays, T-cells (10⁵ cells per well) were stimulated with equal numbers of C1R-HLA-2 or C1R-HLA-2-MICA transfectants pulsed with the pp65 peptide at the indicated concentrations, in the presence or absence of mAb 6D4, mAb 1D11, or control Ig. In the mAb triggering experiments, the T-cells were stimulated with solid-phase anti-CD3 (OKT3; Orthobiotech) with or without mAb 1D11 or control Ig. Antibodies were plate-bound by precoating 96-well flat bottom microtiter plates with goat anti-mouse Fc-specific F(ab′)₂ Ig (Jackson Immunoresearch Laboratories). T-cell supernatants from triplicate wells were harvested and pooled after 24 and 48 h of culture, and the amounts of secreted IFN-γ, TNF-α, GM-CSF, IL-2 and IL-4 were determined by commercial ELISA with matched antibody in relation to cytokine standard pairs (R & D Systems).

T-cell proliferation was measured with rested T-cell clones (10⁵ cells per well; 14-21 days after stimulation) or with freshly isolated peripheral blood CD8/CD28⁻ αβ T-cells after activation with plate-bound mAbs as described above. Cultures were pulsed with [³H]thymidine on day 3 and harvested 16 h later using a Micromate cell harvester (Packard). Incorporated radioactivity was determined using Unifilter GF/C plates and a Topcount (Packard).

HLA-A2 tetramer and intracellular cytokine staining of CMV pp65-specific T-cells from peripheral blood. The HLA-A2-peptide complex tetramers were produced similar to the original method (Altman et al., 1996); Callan et al., 1998). In brief, the extracellular domains of HLA-A2 with a carboxyterminal BirA enzyme substrate site and β₂-microglobulin (β₂m) were expressed in bacteria and purified from inclusion bodies. Complexes of HLA-A2, β₂m and pp65 peptide NLVPMVATV were refolded in vitro in the presence of protease inhibitors, biotinylated and HPLC-purified. Tetramers were obtained by treatment with streptavidin-PE at a molar ratio of 4:1. CD8 αβ T-cells were isolated from peripheral blood of a healthy donor previously typed for HLA-A2 and screened for high numbers of pp65-specific T-cells, using negative selection with RosetteSep™ (StemCell Technologies). T-cells (2×10⁶; >98% CD8 αβ T-cells) were stimulated with equal numbers of C1R-HLA-A2 or C1R-HLA-A2-MICA cells pulsed with the pp65 peptide (500 nM) in the presence of Monensin (0.6 μl/ml; Golgistop, Pharmingen) in 96-well round bottom plates (0.2×10⁶ cells/well) for 8 h at 37° C. Thereafter, pp65-specific-T-cells were identified by staining with the PE-conjugated tetramer reagent, stained with anti-CD28-FITC (Immunotech), fixed and permeabilized using a Cytofix/Cytoperm Plus Kit (Pharmingen), and stained for intracellular IL-2 with an allophycocyanin (APC)-conjugated mAb (Pharmingen). Cells were analyzed with a Becton-Dickinson FACS Vantage cytometer.

### EXAMPLE 2

### Results

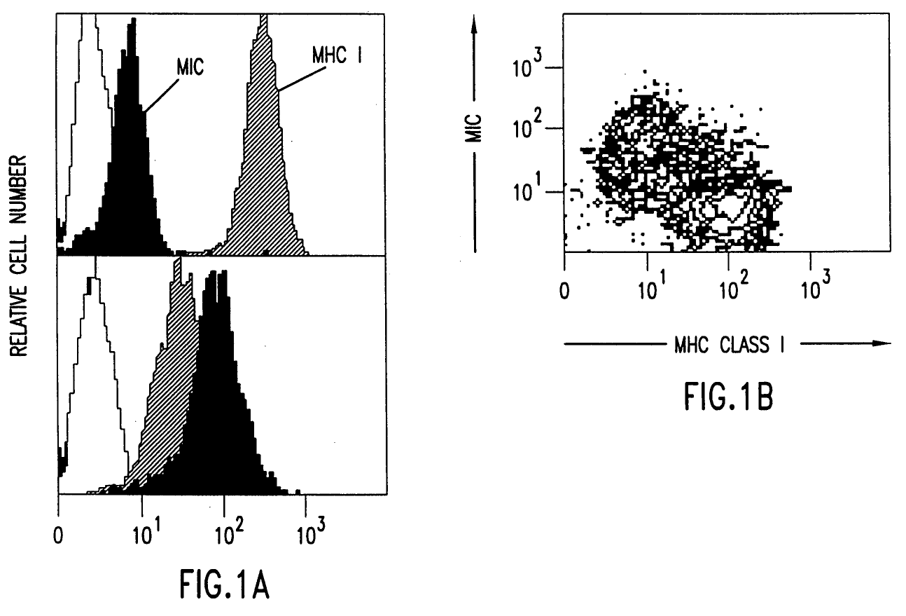

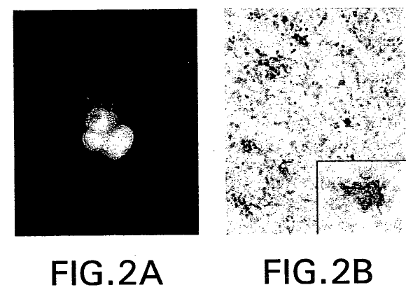

Induction of MIC expression by CMV infection. Surface expression of MIC was monitored on human fibroblasts infected at high multiplicity with the CMV strain AD169 using the monoclonal antibody (mAb) 6D4, which is specific for MICA and MICB, and flow cytometry (Groh et al. 1998). From 24 to 72 h after infection, surface MIC increased progressively to amounts that were about 10-fold higher than those on mock-infected control cells. Concurrently, expression of MHC class I decreased by a similar factor (FIG. 1A). Productive infection of all fibroblasts was confirmed by staining for the CMV immediate-early nuclear antigen-1 (IE-1); moreover, expression of MIC was not induced by UV-inactivated virus, which can enter cells but cannot productively infect (data not shown). Similar results were obtained with endothelial cells, which was physiologically significant since endothelium is a well established site of CMV infection in a chronically infected host. Contour profiles of endothelial cell cultures that were incompletely infected with the viral strain VHL/e at low multiplicity displayed two cell populations with inversely correlated expression levels of MIC and MHC class I (FIG. 1B). Two-color immunostainings of the partially infected endothelial cell monolayers demonstrated that induction of MIC was strictly associated with expression of viral IE-1 (FIG. 2A). These results show that productive infection by different CMV strains potently increases the expression of MIC, presumably as a consequence of the cell stress response. Induction of MIC by CMV was confirmed in vivo, by two-color immunohistochemistry stainings of lung sections from patients with CMV interstitial pneumonia. All of three samples examined included multiple foci of cytomegalic cells that exhibited intense staining for both the CMV delayed-early DNA-binding protein p52 and MIC (FIG. 2B). This observation extended the results obtained in cell culture and supported the physiological significance of the virus-induced expression of MIC.

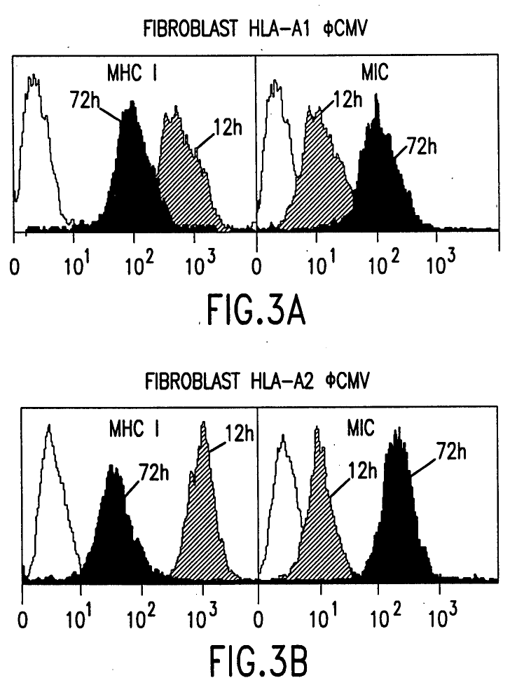

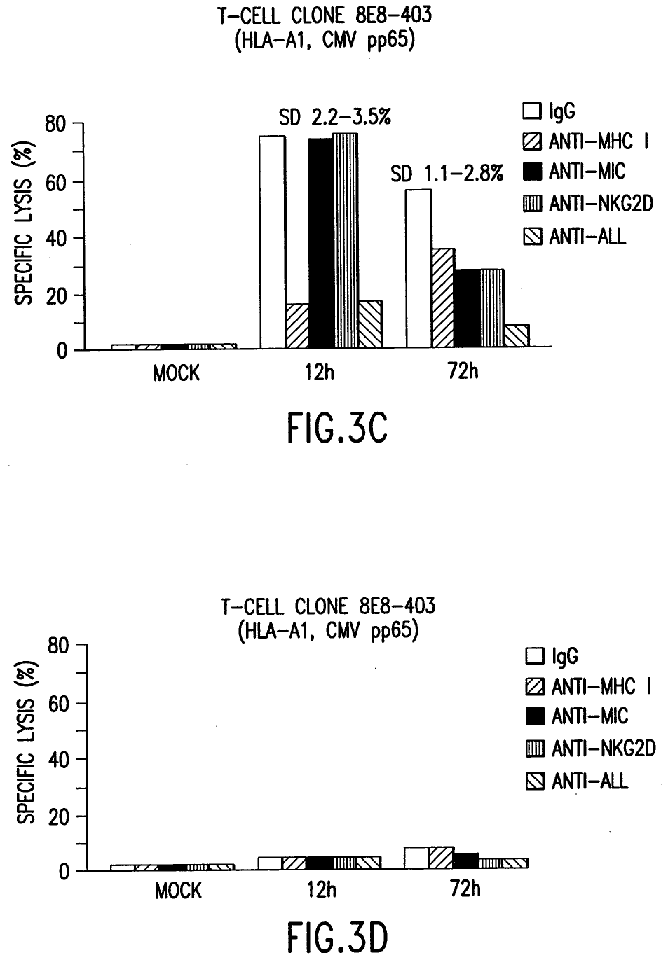

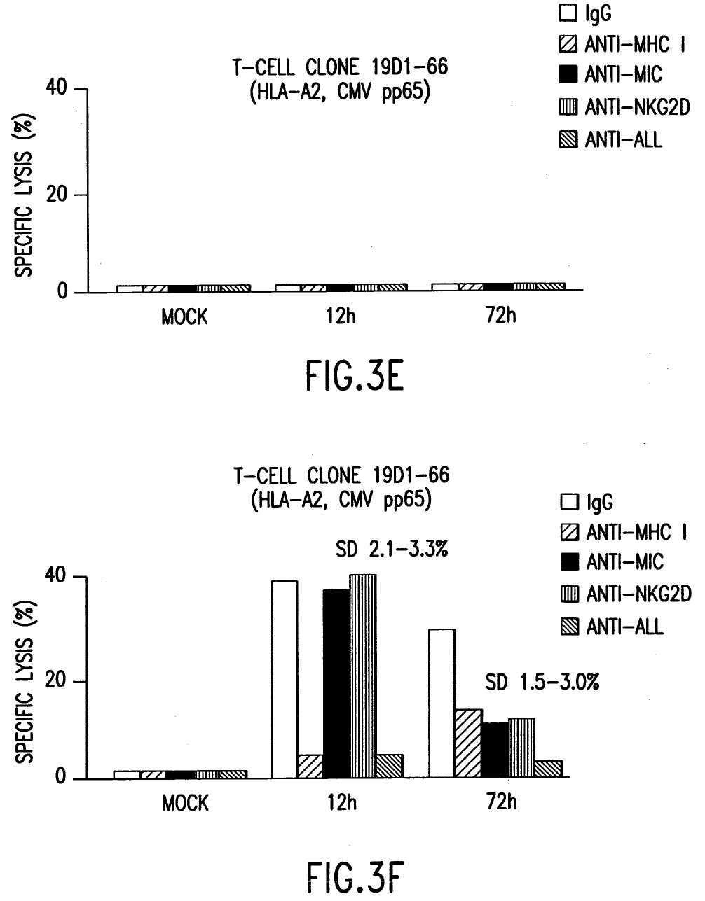

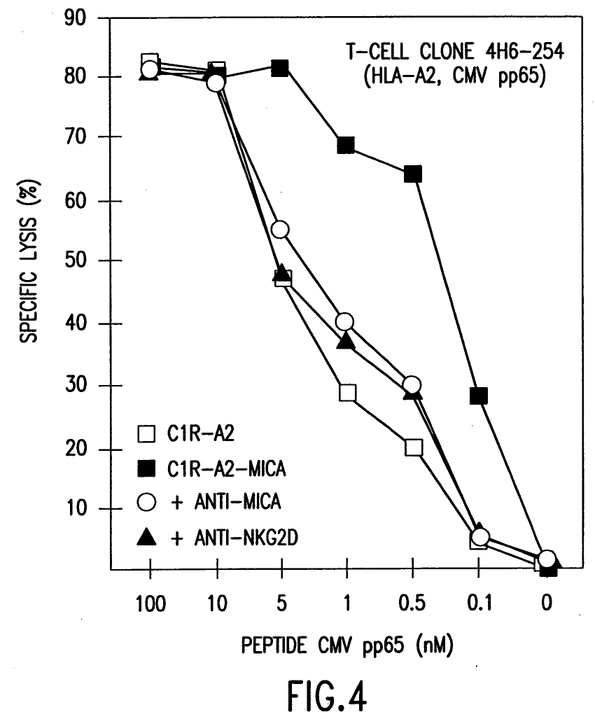

NKG2D-MIC interaction augments cytolytic responses. Although CMV gene products severely impair MHC class I antigen processing and expression, the virus is under immunological control as reflected by the frequent reactivation of CMV and progression to fatal disease in immunocompromised patients (Riddell et al., 1992; Riddell, 1995). Hence, the inventors investigated whether the induced expression of MIC could compensate for deficient MHC class I function, by positively modulating viral antigen-specific CD8 αβ T-cell responses via engagement of NKG2D. This notion was based on the ability of NKG2D to function as an activating receptor in antibody-dependent cytotoxicity assays, although its contribution, if any, to TCR-dependent T-cell activation is unknown (Bauer et al., 1999). A total of nine CD8 αβ T-cell clones (all CD28⁻, CD94⁻, NKG2D⁺; KIR2DL1⁻, KIR2DL2⁻, KIR2DL3⁻; KIR2S1⁻KIR2S2⁻; KIR3DL1⁻, KIR3DL2⁻), which recognize defined epitopes of the CMV pp65 matrix protein in the context of HLA-A1 or -A2 (McLaughlin-Taylor, 1994; Gilbert et al., 1996), were tested in cytotoxicity assays using autologous or HLA-matched fibroblasts infected with CMV AD169 as targets. At 12 h post-infection, a time point at which the surface levels of MHC class I and MIC were yet unchanged (FIG. 3A & 3B), T-cell cytotoxicity was maximal and could be inhibited by mAb against MHC class I (mAb W6/32; pan anti-HLA-A, -B and -C; Parham et al. 1979) but not by mAbs specific for MIC (mAb 6D4; Groh et al., 1998) or NKG2D (mAb 1D11; Bauer et al., 1999) (FIGS. 3C & 3F). Thus, under the conditions of undiminished MHC class I and low MIC expression, NKG2D was not involved in cytolytic T-cell function. By contrast, at 72 h post-infection, when MHC class I expression was impaired and MIC reached maximum surface levels (FIGS. 3A & 3B), mAb masking of MIC or NKG2D substantially reduced target cell lysis (FIGS. 3C & 3F). This was not due to TCR-independent activation resulting from the increased expression of MIC and triggering of NKG2D since no cytotoxicity was observed when T-cell clones were tested against HLA-mismatched virus-infected fibroblasts (FIGS. 3D & 3E). Moreover, mAb masking of MHC class I, MIC and NKG2D altogether was additive in lysis inhibition (FIGS. 3C & 3F). Hence, these results suggested that engagement of NKG2D augmented CMV-specific cytotoxic T-cell responses under conditions of suboptimal MHC—antigen stimulation of TCR. This was confirmed using C1R cell transfectants expressing HLA—A2 alone or together with MICA, which were pulsed with titered concentrations of the CMV pp65 peptide and tested against five of the antigen-specific T-cell clones. At optimal peptide concentrations, both target cell lines were lysed equally well and mAb against MICA or NKG2D. had no inhibitory effects (FIG. 4). However, with increasingly limiting peptide concentrations, the responses against C1R-A2-MICA cells remained substantially stronger than those against the targets lacking MICA, which declined rapidly. This functional augmentation was abrogated by mAbs against MICA or NKG2D and was qualitatively similar to the differences observed with the CMV-infected fibroblasts late versus early after infection. Altogether, these results indicated that NKG2D could enhance anti-CMV and presumably other cytotoxic CD8 αβ T-cell responses.

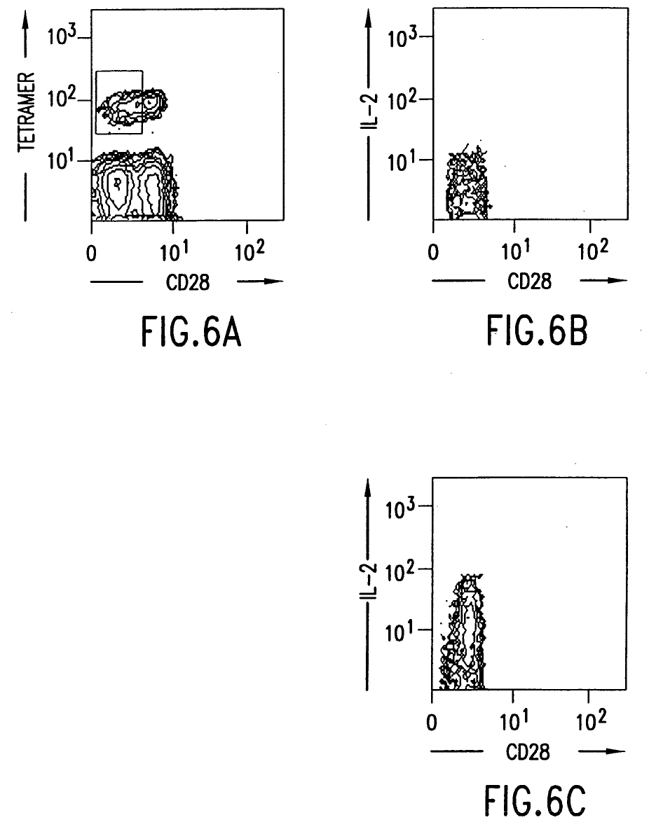

T-cell costimulation by NKG2D. The inventors' observations, together with previous data indicating that NKG2D may signal via its adaptor protein DAP10 in a similar pathway as CD28, raised the question of whether NKG2D could costimulate T-cell activation, by induction of cytokine production and T-cell proliferation. Peptide-pulsed C1R-A2-MICA cells were substantially more potent stimulators (100-500%) of interferon-γ (IFN-γ), tumor necrosis factor-α(TNF-α), IL-4, and granulocyte/macrophage-colony stimulating factor (GM-CSF) release by the A2-restricted pp65-specific T-cell clones than identically treated C1R-A2 cells lacking MICA (FIG. 5A, 5B & 5D, and data not shown). These results were highly reproducible in three independent experiments and were representative of five different T-cell clones tested. Distinct from the results obtained with the cytotoxicity assays, the cytokine responses were superinduced even when MHC-antigen stimulation of TCR by C1R-A2 cells pulsed with saturating peptide concentrations (10-100 nM) was optimal. CD28⁻/CD8 αβ T-cells, the phenotype common to all of the T-cells used in this study so far, fail to produce IL-2 in response to triggering of TCR-CD3 (Azuma et al. 1993). Hence, it was of particular interest that expression of MICA on the stimulator cells resulted in induction of IL-2, which was not detectably produced by T-cells exposed to the MICA-negative cells (FIG. 5C). In all of these experiments, mAb masking of MICA abrogated the augmentation or de novo induction of cytokine production. By contrast, in the presence of anti-NKG2D mAb, the amounts of cytokines were either variably increased or unchanged (data not shown). Thus, in these long-term (24-48 h) cytokine release assays, the anti-NKG2D mAb had at least weak stimulatory capacity, either via binding to NKG2D in solution or after becoming crosslinked, or both. This was opposite to the inhibitory effect of the same soluble mAb in the short-term (4 h) cytotoxicity assays, presumably because the previously observed high affinity interactions of MIC with NKG2D were critical in enhancing effector-target cell contacts and in triggering cytotoxicity (Bauer et al. 1999). The cytokine release observations made with the five T-cell clones could be replicated with CMV-specific CD28⁻/CD8 αβ T-cells identified by staining with HLA-A2-peptide pp65 tetramers among freshly isolated peripheral blood CD8⁺ T-cells (FIG. 6A). After short-term antigen stimulation in the presence but not in the absence of MIC, a proportion of these T-cells showed positive staining for intracellular IL-2 (FIG. 6B & 6C). Collectively, these results clearly supported a costimulatory function of NKG2D.

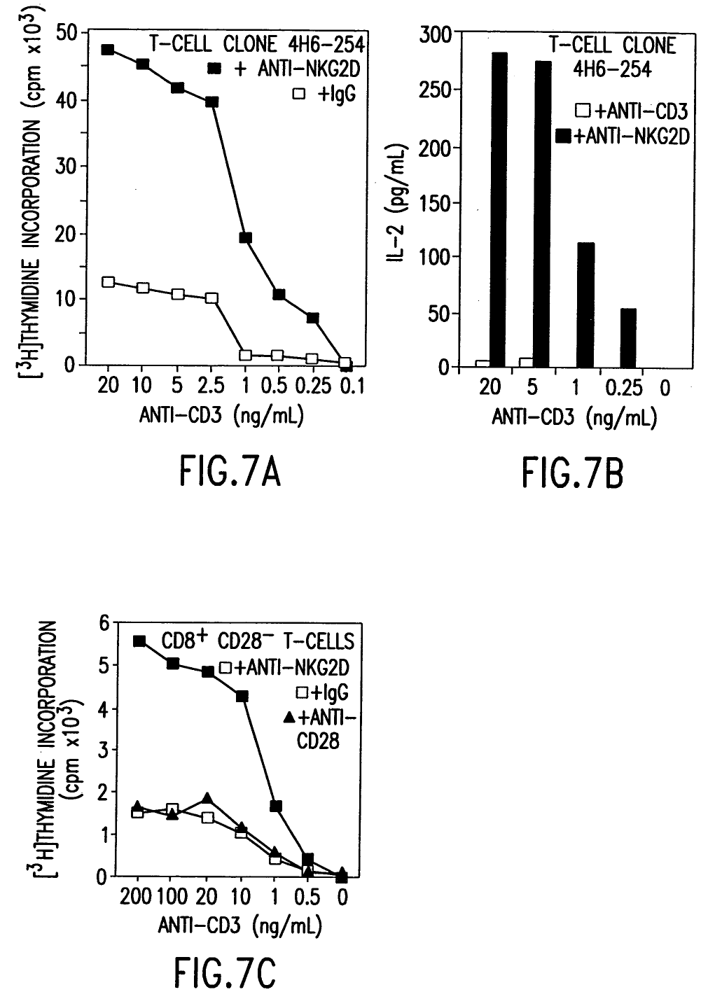

Further evidence for costimulation of CD28⁻/CD8 αβ T-cells by NKG2D was obtained using titered concentrations of solid-phase anti-CD3 with or without anti-NKG2D mAb to stimulate cytokine secretion and proliferation by the pp65-specific T-cells. All of four T-cell clones tested produced no or little IL-2 and IL-4 and showed modest dose-dependent proliferative responses upon triggering with anti-CD3 mAb alone. In the additional presence of anti-NKG2D, however, IL-2 and IL-4 were potently induced and T-cell proliferation was about four-fold amplified (FIGS. 7A & 7B, and data not shown). No effect was seen when anti-NKG2D was used in the absence of anti-CD3. A similar synergistic induction of proliferation was recorded with freshly isolated peripheral blood CD28⁻/CD8⁺ T-cells (FIG. 7C). Thus, NKG2D was a potent costimulator of TCR-CD3 complex-dependent T-cell activation capable of substituting for CD28.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

Almendro,Bellon, Rius, Lastres, Langa, Corbi, Bemabeu, “Cloning of the human platelet endothelial cell adhesion molecule-1 promoter and its tissue-specific expression. Structural and functional characterization,” J. Immunol., 157(12):5411-5421, 1996.

Altman et al., Phenotypic analysis of antigen-specific T lymphocytes. Science 274, 94-98, 1996

Angel, Bauman, Stein, Dellus, Rahmsdorf, and Herrlich, “12-0-tetradecanoyl-phorbol-13-acetate Induction of the Human Collagenase Gene is Mediated by an Inducible Enhancer Element Located in the 5′ Flanking Region,” Mol Cell. Biol., 7:2256, 1987a.

Angel, Imagawa, Chiu, Stein, Imbra, Rahmsdorf, Jonat, Herrlich, and Karin, “Phorbol Ester-Inducible Genes Contain a Common cis Element Recognized by a TPA-Modulated Trans-acting Factor,” Cell, 49:729, 1987b.

Atchison and Perry, “Tandem Kappa Immunoglobulin Promoters are Equally Active in the Presence of the Kappa Enhancer: Implications for Model of Enhancer Function,” Cell, 46:253, 1986.

Atchison and Perry, “The Role of the Kappa Enhancer and its Binding Factor NF-kappa B in the Developmental Regulation of Kappa Gene Transcription,” Cell, 48:121, 1987.

Ausubel et al., In: Current Protocols in Molecular Biology, John, Wiley and Sons, Inc., 1994.

Azuma et al., CD28⁻ T lymphocytes-antigenic and functional properties. J. Immunol., 150, 1147-1159, 1993.

Bahram et al., Proc. Nat'l Acad. Sci. USA, 91:6259-6263, 1994.

Bahram and Spies, T Immunogenetics, 43:230-233, 1996.

Bahram and Spies, T., Nucleotide sequence of a human MHC class I MICB cDNA. Immunogenetics 43, 230-233, 1996.

Baichwal and Sugden, In: Kucherlapati R, ed., Gene Transfer. New York: Plenum Press, pp. 117-148, 1986.

Bakker et al., Killer cell inhibitory receptors for MHC class I molecules regulate lysis of melanoma cells mediated by NK cells, γδ T cells, and antigen-specific CTL. J. Immunol., 160, 5239-5245, 1998.

Banerji et al., “Expression of a Beta-Globin Gene is Enhanced by Remote SV40 DNA Sequences,” Cell, 27:299, 1981.

Banerji, Olson, and Schaffner, “A lymphocyte-specific cellular enhancer is located downstream of the joining region in immunoglobulin heavy-chain genes,” Cell, 35:729, 1983.

Bauer et al., Science 285: 727-729, 1999.

Berkhout et al., “Tat trans-activates the human immunodeficiency virus through a nascent RNA target,” Cell, 59:273, 1989.

Blanar, Baldwin, Flavell, Sharp, “A gamma-interferon-induced factor that binds the interferon response sequence of the MHC class I gene, H-2Kb,” EMBO J., 8(4):1139-44, 1989.

Blomer et al., J. Virol., 71(9): 6641-6649, 1997.

Bodine and Ley, “An enhancer element lies 3′ to the human A gamma globin gene,” EMBO J., 6:2997, 1987.

Boshart, Weber, Jahn, Dorsch-Hasler, Fleckenstein, and Schaffner, “A very strong enhancer is located upstream of an immediate early gene of human cytomegalovirus,” Cell, 41:521, 1985.

Bosze, Thiesen, and Charnay, “A transcriptional enhancer with specificity for erythroid cells is located in the long terminal repeat of the friend murine leukemia virus,” EMBO J., 5:1615, 1986.

Braddock, Chambers, Wilson, Esnouf, Adams, Kingsman, and Kingsman, “HIV-I Tat activates presynthesized RNA in the nucleus,” Cell, 58:269, 1989.

Bulla and Siddiqui, “The hepatitis B virus enhancer modulates transcription of the hepatitis B virus surface-antigen gene from an internal location,” J. Virol., 62:1437, 1986.

Campbell and Villarreal, “Functional analysis of the individual enhancer core sequences of polyoma virus: cell-specific uncoupling of DNA replication from transcription,” Mol. Cell. Biol., 8:1993, 1988.

Campbell, In: Monoclonal Antibody Technology, Laboratory Techniques in Biochemistry and Molecular Biology, Vol. 13, Burden and Von Knippenberg, Eds. pp. 75-83, Amsterdam, Elseview, 1984.

Campere and Tilghman, “Postnatal repression of the α-fetoprotein gene is enhancer independent,” Genes and Dev., 3:537, 1989.

Campo, Spandidos, Lang, Wilkie, “Transcriptional control signals in the genome of bovine papilloma virus type 1, ” Nature, 303:77, 1983.

Capaldi et al., Biochem. Biophys. Res. Comm., 76:425, 1977.

Carbonelli, Corley, Seigelchifer, Zorzopulos, “A plasmid vector for isolation of strong promoters in E. coli,” FEMS Microbiol Lett. 177(1):75-82, 1999.

Carena et al., Major histocompatibility complex class I molecules modulate activation threshold and early signaling of T cell antigen receptor-γδ stimulated by nonpeptide ligands. J. Exp. Med., 186, 1769-1774, 1997.

Celander and Haseltine, “Glucocorticoid regulation of murine leukemia virus transcription elements is specified by determinants within the viral enhancer region,” J. Virology, 61:269, 1987.

Celander, Hsu, and Haseltine, “Regulatory Elements Within the Murine Leukemia Virus Enhancer Regions Mediate Glucocorticoid Responsiveness,” J. Virology, 62:1314, 1988.

Cerwenka et al., Retinoic acid early inducible genes define a ligand family for the activating NKG2D receptor in mice. Immunity, 12, 721-727, 2000.

Chalupny et al., Soluble forms of the novel MHC class I-related molecules ULBP1 and ULBP2 bind to, and functionally activate NK cells. FASEB J. 14, A1018, 2000

Chambers, C. A. & Allison, J. P., Costimulatory regulation of T cell function. Curr. Opin. Cell Biol., 11, 203-210, 1999.

Chandler, Maler, and Yamamoto, “DNA Sequences Bound Specifically by Glucocorticoid Receptor in vitro Render a Heterlogous Promoter Hormone Responsive in vivo,” Cell, 33:489, 1983.

Chandler, Mayeda, Yeakley, Krainer, Fu, “RNA splicing specificity determined by the coordinated action of RNA recognition motifs in SR proteins,” Proc Nat'l Acad. Sci. U.S.A., 94(8):3596-3601, 1997.

Chang, Erwin, and Lee, “Glucose-regulated Protein (GRP94 and GRP78) Genes Share Common Regulatory Domains and are Coordinately Regulated by Common Trans-acting Factors,” Mol. Cell. Biol., 9:2153, 1989.

Chatterjee, Lee, Rentoumis, and Jameson, “Negative Regulation of the Thyroid-Stimulating Hormone Alpha Gene by Thyroid Hormone: Receptor Interaction Adjacent to the TATA Box,” Proc Nat'l . Acad Sci. U.S.A., 86:9114, 1989.

Chen and Okayama, Mol. Cell Biol., 7:2745-2752, 1987.

Choi, Chen, Kriegler, and Roninson, “An altered pattern of cross-resistance in multi-drug-resistant human cells results from spontaneous mutations in the mdr-1 (p-glycoprotein) gene,” Cell, 53:519, 1988.

Cocea, “Duplication of a region in the multiple cloning site of a plasmid vector to enhance cloning-mediated addition of restriction sites to a DNA fragment,” Biotechniques, 23:814-816, 1997.

Cohen, Walter, and Levinson, “A Repetitive Sequence Element 3′ of the Human c-Ha-ras1 Gene Has Enhancer Activity,” J. Cell. Physiol., 5:75, 1987.

Costa, Lai, Grayson, and Darnell, “The Cell-Specific Enhancer of the Mouse Transthyretin (Prealbumin) Gene Binds a Common Factor at One Site and a Liver-Specific Factor(s) at Two Other Sites,” Mol Cell. Biol., 8:81, 1988.

Cotton, R. G. H., Biochem J., 263:1-10, 1989.

Coupar et al., “A general method for the construction of recombinant vaccinia virus expressing multiple foreign genes,” Gene, 68:1-10, 1988.

Craig, E. A. & Gross, C. A., Trends Biochem. Sci., 16,135-139, 1991.

Cripe, Haugen, Turk, Tabatabai, Schmid, Durst, Gissmann, Roman, and Turek, “Transcriptional Regulation of the Human Papilloma Virus-16 E6-E7 Promoter by a Keratinocyte-Dependent Enhancer, and by Viral E2 Trans-Activator and Repressor Gene Products: Implications for Cervical Carcinogenesis,” EMBO J., 6:3745, 1987.

Culotta and Hamer, “Fine Mapping of a Mouse Metallothionein Gene Metal-Response Element,” Mol. Cell. Biol., 9:1376, 1989.

Curiel, “Gene transfer mediated by adenovirus-polylysine DNA complexes,” In: Viruses in Human Gene Therapy, J. -M. H. Vos (Ed.), Carolina Academic Press, Durham,. N.C., pp 179-212, 1994.

Dandolo, Blangy, and Kamen, “Regulation of Polyma Virus Transcription in Murine Embryonal Carcinoma Cells,” J. Virology, 47:55, 1983.

Davis et al., Ligand recognition by αβ T cell receptors. Annu. Rev. Immunol. 16, 523-544,-1998

De Villiers et al., “Polyoma Virus DNA Replication Requires an Enhancer,” Nature, 312:242, 1984.

Deschamps, Meijlink, and Verma, “Identification of a Transcriptional Enhancer Element Upstream From the Proto-Oncogene Fos,” Science, 230:1174, 1985.

Diefenbach et al., “Ligands for the murine NKG2D receptor: expression by tumor cells and activation of NK cells and macrophages”, Nat. Immunol. 1, 119-126, 2000.

Edbrooke, Burt, Cheshire, and Woo, “Identification of cis-acting sequences responsible for phorbol ester induction of human serum amyloid a gene expression via a nuclear-factor-kappa β-like transcription factor,” Mol. Cell. Biol., 9:1908, 1989.

Edlund, Walker, Barr, and Rutter, “Cell-specific expression of the rat insulin gene: evidence for role of two distinct 5′ flanking elements,” Science, 230:912, 1985.

EPO 0273085

Fechheimer et al., Proc. Nat'l Acad. Sci. USA, 84:8463-8467, 1987.

Feng and Holland, “HIV-I Tat Trans-Activation Requires the Loop Sequence Within Tar,” Nature, 334:6178, 1988.

Firak and Subramanian, “Minimal Transcription Enhancer of Simian Virus 40 is a 74-Base-Pair Sequence that Has Interacting Domains,” Mol. Cell. Biol., 6:3667, 1986.

Foecking and Hofstetter, “Powerful and Versatile Enhancer-Promoter Unit for Mammalian Expression Vectors,” Gene, 45(1):101-105, 1986.

Fraley et al., Proc. Nat'l Acad. Sci. USA, 76:3348-3352, 1979.

Friedmann, “Progress toward human gene therapy,” Science, 244:1275-1281, 1989.

Fujita, Shibuya, Hotta, Yamanishi, and Taniguchi, “Interferon-Beta Gene Regulation: Tandemly Repeated Sequences of a Synthetic 6-bp Oligomer Function as a Virus-Inducible Enhancer,” Cell, 49:357, 1987.

Gefter et al., Somatic Cell Genet., 3: 231-236, 1977.

Germain, R. N. & Margulies, D. H., The biochemistry and cell biology of antigen processing and presentation. Annu. Rev. Immunol. 11, 403-450, 1993.

Ghosh and Bachhawat, In: Wu G. and C. Wu ed. Liver diseases, targeted diagnosis and therapy using specific receptors and ligands. New York: Marcel Dekker, pp. 87-104, 1991.

Ghosh-Choudhury et al., EMBO J., 6:1733-1739, 1987.

Gibbs, R. A. and Caskey, T. C., Science 236: 303-305, 1987.

Gilbert et al., Cytomegalovirus selectively blocks antigen processing and presentation of its immediate-early gene product. Nature 383, 720-722, 1996.

Gilles, Morris, Oi, and Tonegawa, “A tissue-specific transcription enhancer element is located in the major intron of a rearranged immunoglobulin heavy-chain gene,” Cell, 33:717, 1983.

Gimmi et al., “B-cell surface antigen B7 provides a costimulatory signal that induces T cells to proliferate and secrete interleukin 2,” Proc. Nat 7 Acad. Sci. USA 88, 6575-6579, 1991.

Gloss, Bernard, Seedorf, and Klock, “The Upstream Regulatory Region of the Human-Papilloma Virus-16 Contains an E2 Protein-Independent Enhancer Which is Specific for Cervical Carcinoma Cells and Regulated by Glucocorticoid Hormones,” EMBO J., 6:3735, 1987.

Godbout, Ingram, and Tilghman, “Fine-Structure Mapping of the Three Mouse Alpha-Fetoprotein Gene Enhancers,” Mol. Cell. Biol., 8:1169, 1988.

Goding, In Monoclonal Antibodies: Principles and Practice, 2d ed., Orlando, Fla., Academic Press, 1986, pp. 60-61, and 71-74, 1986.

Goodbourn and Maniatis, “Overlapping Positive and Negative Regulatory Domains of the Human β-Interferon Gene,” Proc. NatL. Acad. Sci. USA, 85:1447, 1988.

Goodbourn, Burstein, and Maniatis, “The Human Beta-Interferon Gene Enhancer is Under Negative Control,” Cell, 45:601, 1986.

Gopal, Mol. Cell Biol., 5:1188-1190, 1985

Graham and van der Eb, Virology, 52:456-467, 1973

Greene, Bohnlein, and Ballard, “HIV-1, and Normal T-Cell Growth: Transcriptional Strategies and Surprises,” Immunology Today, 10:272, 1989

Gribben et al., “CTLA-4 mediates antigen-specific apoptosis of human T cells,” Proc. Nat'l Acad. Sci. USA, 92, 811-815, 1995.

Groh et al., “Broad tumor-associated expression and recognition by tumor-derived γδ T cells of MICA and MICB,” Proc. Nat'l Acad. Sci. USA, 96, 6879-6884, 1999.

Groh et al., “Cell stress-regulated human major histocompatibility complex class I gene expressed in gastrointestinal epithelium,” Proc. Nat 'l Acad. Sci. USA, 93, 12445-12450, 1996

Groh, V., Steinle, A., Bauer, S. & Spies, T. “Recognition of stress-induced MHC molecules by intestinal epithelial γδ T cells,”. Science, 279, 1737-1740, 1998.

Grosschedl and Baltimore, “Cell-Type Specificity of Immunoglobulin Gene Expression is Regulated by at Least Three DNA Sequence Elements,” Cell, 41:885, 1985.

Grunhaus and Horwitz, Seminar in Virology, 3:237-252, 1992.

Hara et al., Human T cell activation. II. A new activation pathway used by a major T cell population via a disulfide-bonded dimer of a 44 kilodalton polypeptide (9.3 antigen). J. Exp. Med. 161, 1513-1524, 1985

Harding et al., CD28-mediated signalling co-stimulates murine T cells and prevents induction of anergy in T-cell clones. Nature 356, 607-609, 1992.

Harland & Weintraub, J. Cell Biol., 101:1094-1099, 1985.

Haslinger and Karin, “Upstream Promoter Element of the Human Metallothionein-II Gene Can Act Like an Enhancer Element,” Proc. Natl. Acad. Sci. U.S.A., 82:8572, 1985.

Hauber and Cullen, “Mutational Analysis of the Trans-Activiation-Responsive Region of the Human Immunodeficiency Virus Type I Long Terminal Repeat,” J. Virology, 62:673, 1988.

Hen, Borrelli, Fromental, Sassone-Corsi, and Chambon, “A Mutated Polyoma Virus Enhancer Which is Active in Undifferentiated Embryonal Carcinoma Cells is not Repressed by Adenovirus-2 EIA Products,” Nature, 321:249, 1986.

Hensel, Meichle, Pfizenmaier, and Kronke, “PMA-Responsive 5° Flanking Sequences of the Human TNF Gene,” Lymphokine Res., 8:347, 1989.

Herr and Clarke, “The SV40 Enhancer is Composed of Multiple Functional Elements That Can Compensate for One Another,” Cell, 45:461,1986.

Hirochika, Browker, and Chow, “Enhancers and Trans-Acting E2 Transcriptional Factors of Papilloma Viruses,” J. Virol., 61:2599, 1987.

Hirsch, Gaugler, Deagostini-Bauzin, Bally-Cuif, and Gordis, “Identification of Positive and Negative Regulatory Elements Governing Cell-Type-Specific Expression of the Neural-Cell-Adhesion-Molecule Gene,” Mol. Cell. Biol., 10:1959, 1990.

Holbrook, Gulino, and Ruscetti, “cis-Acting Transcriptional Regulatory Sequences in the Gibbon Ape Leukemia Virus (GALV) Long Terminal Repeat,” Virology, 157:211, 1987.

Horlick and Benfield, “The Upstream Muscle-Specific Enhancer of the Rat Muscle Creatine Kinase Gene is Composed of Multiple Elements,” Mol. Cell. Biol., 9:2396, 1989.

Horwich et al. J. Virol., 64:642-650, 1990.

Huang, Ostrowski, Berard, and Hagar, “Glucocorticoid regulation of the ha-musv p21 gene conferred by sequences from mouse mammary tumor virus,” Cell, 27:245, 1981.

Hug, Costas, Staeheli, Aebi, and Weissmann, “Organization of the Murine Mx Gene and Characterization of its Interferon- and Virus-Inducible Promoter,” Mol Cell. Biol., 8:3065, 1988.

Hwang, Lim, and Chae, “Characterization of the S-Phase-Specific Transcription Regulatory Elements in a DNA-Replication-Independent Testis-Specific H2B (TH2B) Histone Gene,” Mol. Cell. Biol., 10:585, 1990.

Ikeda et al., Characterization of an antigen that is recognized on a melanoma showing partial HLA loss by CTL expressing an NK inhibitory receptor,” Immunity, 6, 199-208, 1997.

Imagawa, Chiu, and Karin, “Transcription Factor AP-2 Mediates Induction by Two Different Signal-Transduction Pathways: Protein Kinase C and cAMP,” Cell, 51:251, 1987.

Imbra and Karin, “Phorbol Ester Induces the Transcriptional Stimulatory Activity of the SV40 Enhancer,” Nature, 323:555, 1986.

Imler, Lemaire, Wasvlyk, and Waslyk, “Negative Regulation Contributes to Tissue Specificity of the Immunoglobulin Heavy-Chain Enhancer,” Mol. Cell. Biol, 7:2558, 1987.

Imperiale and Nevins, “Adenovirus 5 E2 Transcription Unit: an E1A-Inducible Promoter with an Essential Element that Functions Independently of Position or Orientation,” Mol. Cell. Biol., 4:875, 1984.

Inouye et al., “Up-promoter mutations in the lpp gene of Escherichia coli,” Nucl. Acids Res., 13:3101-3109, 1985.

Jakobovits, Smith, Jakobovits, and Capon, “A Discrete Element 3′ of Human Immunodeficiency Virus 1 (HIV-1) and HIV-2 mRNA Initiation Sites Mediates Transcriptional Activation by an HIV Trans-Activator,” Mol. Cell. Biol., 8:2555, 1988.

Jameel and Siddiqui, “The Human Hepatitis B Virus Enhancer Requires Transacting Cellular Factor(s) for Activity,” Mol. Cell. Biol., 6:710, 1986.

Jaynes, Johnson, Buskin, Gartside, and Hauschka, “The Muscle Creatine Kinase Gene is Regulated by Multiple Upstream Elements, Including a Muscle-Specific Enhancer,” Mol. Cell. Biol., 8:62, 1988.

Johannesson et al., “Bicyclic tripeptide mimetics with reverse turn inducing properties.” J. Med. Chem., 42:601-608, 1999.

Johnson et al., In BIOTECHNOLOGYAND PHARMACY, Pezzuto et al., Eds., Chapman and Hall, New York 1993.

Johnson, Wold, and Hauschka, “Muscle creatine kinase sequence elements regulating skeletal and cardiac muscle expression in transgenic mice,” Mol. Cell. Biol., 9:3393, 1989.

Kadesch and Berg, “Effects of the Position of the Simian Virus 40 Enhancer on Expression of Multiple Transcription Units in a Single Plasmid,” Mol. Cell. Biol., 6:2593, 1986.

Kaneda et al., Science, 243:375-378, 1989.

Karin, Haslinger, Heguy, Dietlin, and Cooke, “Metal-Responsive Elements Act as Positive Modulators of Human Metallothionein-IIA Enhancer Activity,” Mol. Cell. Biol., 7:606, 1987.

Katinka, Vasseur, Montreau, Yaniv, and Blangy, “Polyoma DNA Sequences Involved in the Control of Viral Gene Expression in Murine Embryonal Carcinoma Cells,” Nature, 290:720, 1981.

Katinka, Yaniv, Vasseur, and Blangy, “Expression of Polyoma Early Functions in Mouse Embryonal Carcinoma Cells Depends on Sequence Rearrangements in the Beginning of the Late Region,” Cell, 20:393, 1980.

Kato et al., J. Biol. Chem., 266:3361-3364, 1991.

Kawamoto, Makino, Niw, Sugiyama, Kimura, Anemura, Nakata, and Kakunaga, “Identification of the Human Beta-Actin Enhancer and its Binding Factor,” Mol. Cell. Biol., 8:267, 1988.

Kelleher and Vos, “Long-term episomal gene delivery in human lymphoid cells using human and avian adenoviral-assisted transfection,” Biotechniques, 17(6): 1110-1117, 1994.

Kiledjian, Su, Kadesch, “Identification and characterization of two functional domains within the murine heavy-chain enhancer,” Mol. Cell. Biol., 8:145, 1988.

Klamut, Gangopadyhay, Worton, and Ray, “Molecular and Functional Analysis of the Muscle-Specific Promoter Region of the Duchenne Muscular Dystrophy Gene,” Mol. Cell. Biol., 10:193, 1990.

Klein et al., Nature, 327:70-73, 1987

Koch, Benoist, and Mathis, “Anatomy of a new β-cell-specific enhancer,” Mol. Cell. Biol., 9:303, 1989.

Kohler and Milstein, Eur. J. Immunol., 6:511-519, 1976.

Kohler and Milstein, Nature, 256:495-497, 1975.

Kraus et al., “Alternative promoter usage and tissue specific expression of the mouse somatostatin receptor 2 gene,” FEBS Lett., 428(3):165-170, 1998.

Kriegler and Botchan, “A retrovirus LTR contains a new type of eukaryotic regulatory element,” In: Eukaryotic Viral Vectors, Gluzman (ed.), Cold Spring Harbor, Cold Spring Harbor Laboratory, N.Y., 1982.

Kriegler and Botchan, “Enhanced transformation by a simian virus 40 recombinant virus containing a Harvey murine sarcoma virus long terminal repeat,” Mol. Cell. Biol. 3:325, 1983.

Kriegler et al., “Promoter substitution and enhancer augmentation increases the penetrance of the sv40 a gene to levels comparable to that of the harvey murine sarcoma virus ras gene in morphologic transformation,” In: Gene Expression, Alan Liss (Ed.), Hamer and Rosenberg, New York, 1983.

Kriegler et al., “Transformation Mediated by the SV40 T Antigens: Separation of the Overlapping SV40 Early Genes with a Retroviral Vector,” Cell, 38:483, 1984.

Kriegler et al., “Viral Integration and Early Gene Expression Both Affect the Efficiency of SV40 Transformation of Murine Cells: Biochemical and Biological Characterization of an SV40 Retrovirus,” In: Cancer Cells 2/Oncogenes and Viral Genes, Van de Woude et al. eds, Cold Spring Harbor: Cold Spring Harbor Laboratory, 1984.

Kriegler, Perez, Defay, Albert and Liu, “A Novel Form of TNF/Cachectin Is a Cell-Surface Cytotoxix Transmembrane Protein: Ramifications for the Complex Physiology of TNF,” Cell, 53:45, 1988.

Kriegler, Perez, Hardy and Botchan, “Transformation mediated by the sv40 t antigens: separation of the overlapping sv40 early genes with a retroviral vector,” Cell, 38:483, 1984.

Kuhl, De La Fuenta, Chaturvedi, Parinool, Ryals, Meyer, and Weissman, “Reversible Silencing of Enhancers by Sequences Derived From the Human IFN-alpha Promoter,” Cell, 50:1057, 1987.

Kunz, Zimmerman, Heisig, and Heinrich, “Identification of the Promoter Sequences Involved in the Interleukin-6-Dependent Expression of the Rat Alpha-2-Macroglobulin Gene,” Nucl. Acids Res., 17:1121, 1989.

Kyte and Doolittle, J. Mol. Biol., 157(1):105-132, 1982.

Lanier et al., Immunoreceptor DAP12 bearing a tyrosine-based activation motif is involved in activating NK cells. Nature 391, 703-707, 1998

Lanier, L. L., Turning on natural killer cells. J. Exp. Med. 191, 1259-1262, 2000.

Lareyre et al., “A 5-kilobase pair promoter fragment of the murine epididymal retinoic acid-binding protein gene drives the tissue-specific, cell-specific, and androgen-regulated expression of a foreign gene in the epididymis of transgenic mice,” J. Biol. Chem., 274(12):8282-8290, 1999.

Larsen, Harney, and Moore, “Repression medaites cell-type-specific expression of the rat growth hormone gene,” Proc Nat'l. Acad. Sci. USA., 83:8283, 1986.

Laspia, Rice, and Mathews, “HIV-1 Tat protein increases transcriptional initiation and stabilizes elongation,” Cell, 59:283, 1989.

Latimer, Berger, and Baumann, “Highly conserved upstream regions of the alpha..sub.1-antitrypsin gene in two mouse species govern liver-specific expression by different mechanisms,” Mol. Cell. Biol., 10:760, 1990.

Laughlin, Cardellichio, and Coon, “Latent infection of kb cells with adeno-associated virus type 2,” J. Virol, 60:515-524, 1986.

Lebkowski, McNally, Okarma, and Lerch, “Adeno-associated virus: a vector system for efficient introduction and integration. of DNA into a variety of mammalian cell types,” Mol. Cell. Biol., 8:3988-3996, 1988.

Lee et al., “Activation of beta3-adrenoceptors by exogenous dopamine to lower glucose uptake into rat adipocytes,” J. Auton Nerv Syst, 74(2-3):86-90, 1997.

Lee, Mulligan, Berg, and Ringold, .“Glucocorticoids Regulate Expression of Dihydrofolate. Reductase cDNA in Mouse Mammary Tumor Virus Chimaeric Plasmids,” Nature, 294:228, 1981.

Lee, N. et al. HLA-E is the major ligand for the natural killer inhibitory receptor CD94/NKG2A. Proc. Nat'l Acad. Sci. USA, 95, 5199-5204, 1998.

Lenschow et al., “CD28/B7 system of T cell costimulation,” Annu. Rev. Immunol., 14, 233-258, 1996.

Levenson et al., “Internal ribosomal entry site-containing retroviral vectors with green fluorescent protein and drug resistance markers,” Human Gene Therapy, 9:1233-1236, 1998.

Levinson, Khoury, VanDeWoude, and Gruss, “Activation of SV40 Genome by 72-Base-Pair Tandem Repeats of Moloney Sarcoma Virus,” Nature, 295:79, 1982.

Li et al., “Crystal structure of the MHC class I homolog MIC-A, a γδ T cell ligand,” Immunity, 10, 577-584, 1999.

Lin, Cross, Halden, Dragos, Toledano, and Leonard, “Delineation of an enhancerlike positive regulatory element in the interleukin-2 receptor chain gene,” Mol. Cell. Biol., 10:850, 1990.

Linsley et al., Binding of the B cell activation antigen B7 to CD28 costimulates T cell proliferation and interleukin 2 mRNA-accumulation. J. Exp. Med. 173, 721-730, 1991

Long, E. O., “Regulation of immune responses through inhibitory receptors;” Annu. Rev. Immunol., 17, 875-904, 1999.

Luria, Gross, Horowitz, and Givol, “Promoter Enhancer Elements in the Rearranged Alpha-Chain Gene of the Human T-Cell Receptor,” EMBO J., 6:3307, 1987.

Lusky and Botchan, “Transient Replication of Bovine Papilloma Virus Type 1 Plasmids: cis and trans Requirements,” Proc Nat'l. Acad. Sci. USA., 83:3609, 1986.

Lusky, Berg, Weiher, and Botchan, “Bovine Papilloma Virus Contains an Activator of Gene Expression at the Distal End of the Early Transcription Unit,” Mol. Cell. Biol. 3:1108, 1983.

Macejak and Sarnow, “Internal initiation of translation mediated by the 540 leader of a cellular mRNA,” Nature, 353:90-94, 1991.

Majors and Varmus, “A Small Region of the Mouse Mammary Tumor Virus Long Terminal Repeat Confers Glucocorticoid Hormone Regulation on a Linked Heterologous Gene,” Proc. Nat'l. Acad. Sci. USA, 80:5866, 1983.

Mann et al., Cell, 33:153-159, 1983

Mann et al., “Construction of a retrovirus packaging mutant and its use to produce helper-free defective retrovirus,” Cell, 33:153-159, 1983.

McLaughlin, Collis, Hermonat, and Muzyczka, “Adeno-Associated Virus General Transduction Vectors: Analysis of Proviral Structures,” J. Virol., 62:1963-1973, 1988.

McLaughlin-Taylor et al., Identification of the major late human cytomegalovirus matrix protein pp65 as a target antigen for CD8⁺ virus-specific cytotoxic T lymphocytes. J. Med. Virol. 43, 103-110, 1994.

McNeall, Sanchez, Gray, Chesterman, and Sleigh, “Hyperinducible Gene Expression From a Metallotionein Promoter Containing Additional Metal-Responsive Elements,” Gene, 76:81, 1989.

Miksicek, Heber, Schmid, Danesch, Posseckert, Beato, and Schutz, “Glucocorticoid Responsiveness of the Transcriptional Enhancer of Moloney Murine Sarcoma Virus,” Cell, 46:203, 1986.

Miller, Curr. Top. Microbiol. Immunol., 158:1, 1992.

Mordacq and Linzer, “Co-localization of Elements Required for Phorbol Ester Stimulation and Glucocorticoid Repression of Proliferin Gene Expression,” Genes and Dev., 3:760, 1989.

Moreau, Hen, Wasylyk, Everett, Gaub, and Chambon, “The SV40 base-repair repeat has a striking effect on gene expression both in sv40 and other chimeric recombinants,” Nucl. Acids Res., 9:6047, 1981.

Musesing, Smith, and Capon, “Regulation of mRNA Accumulation by a Human Immunodeficiency Virus Trans-Activator Protein,” Cell, 48:691, 1987.

Muzyczka, “Use of Adeno-Associated Virus as a General Transduction Vector for Mammalian Cells,” Curr. Top. Microbiol. Immunol., 158:97-129, 1992.

Nabel et al., “Recombinant gene expression in vivo within endothelial cells of the arterial wall,” Science, 244:1342-1344, 1989.

Ng, Gunning, Liu, Leavitt, and Kedes, “Regulation of the Human Beta-Actin Promoter by Upstream and Intron Domains,” Nuc. Acids Res., 17:601, 1989.

Nicolas and Rubinstein, In: Vectors: A survey ofmolecular cloning vectors and their uses, Rodriguez and Denhardt (eds.), Stoneham: Buttenvorth, pp. 494-513, 1988.

Nicolau and Sene, Biochem. Biophys. Acta, 721:185-190, 1982.

Nomoto et al., “Cloning and characterization of the alternative promoter regions of the human LIMK2. gene responsible for alternative transcripts with tissue-specific expression,” Gene, 236(2):259-271, 1999.

Noppen et al., C-type lectin-like receptors in peptide-specific HLC class I-restricted expression and modulation of effector functions in clones sharing identical TCR structure and epitope specificity. Eur. J. Immunol. 28, 1134-1142, 1998.

Omirulleh et al., “Activity of a chimeric promoter with the doubled CaMV 35S enhancer element in protoplast-derived cells and transgenic plants in maize,” Plant Mol. Biol., 21:415-28, 1993.

Omitz, Hammer, Davison, Brinster, and Palmiter, “Promoter and enhancer elements from the rat elastase i gene function independently of each other and of heterologous enhancers,” Mol. Cell. Biol. 7:3466, 1987.

Ondek, Sheppard, and Herr, “Discrete Elements Within the SV40 Enhancer Region Display Different Cell-Specific Enhancer Activities,” EMBO J., 6:1017, 1987.

Palmiter, Chen, and Brinster, “Differential regulation of metallothionein-thymidine kinase fusion genes in transgenic mice and their offspring,” Cell, 29:701, 1982.

Parham et al., Nature 279: 639-641, 1979.

Parham et al., Use of a monoclonal antibody (W6/32) in structural studies of HLA-A, B, C antigens. J. Immunol. 123, 342-349, 1979.

Paskind et al., Virology, 67:242-248, 1975.

Pech, Rao, Robbins, and Aaronson, “Functional identification of regulatory elements within the promoter region of platelet-derived growth factor 2, ” Mol. Cell. Biol., 9:396, 1989.

Pelletier and Sonenberg, “Internal initiation of translation of eukaryotic mRNA directed by a sequence derived from poliovirus RNA,” Nature, 334:320-325, 1988.

Perales et al., Proc. Nat'l Acad. Sci. USA, 91:4086-4090, 1994.

Perez-Stable and Constantini, “Roles of fetal γ-globin prorrioter elements and the adult β-globin 3′ enhancer in the stage-specific expression of globin genes,” Mol. Cell. Biol., 10:1116, 1990.

Phillips et al., “Superantigen-dependent, cell-mediated cytotoxicity inhibited by MHC class I receptors on T lymphocytes,” Science, 268, 403-405, 1995

Picard and Schaffner, “A lymphocyte-specific enhancer in the mouse immunoglobulin kappa gene,” Nature, 307:83, 1984.

Pinkert, Omitz, Brinster, and Palmiter, “An albumin enhancer located 10 kb upstream functions along with its promoter to direct efficient, liver-specific expression in transgenic mice,” Genes and Dev., 1:268, 1987.

Ponta, Kennedy, Skroch, Hynes, and Groner, “Hormonal Response Region in the Mouse Mammary Tumor Virus Long Terminal Repeat Can Be Dissociated From the Proviral Promoter and Has Enhancer Properties,” Proc. Nat'l. Acad. Sci. USA, 82:1020, 1985.

Porton, Zaller, Lieberson, and Eckhardt, “Immunoglobulin heavy-chain enhancer is required to maintain transfected .gamma.2a gene expression in a pre-b-cell line,” Mol. Cell. Biol., 10:1076, 1990.

Posnett et al., Differentiation of human CD8 T cells: implications for in vivo persistence of CD8⁺ CD28⁻ cytotoxic effector clones. Int. Immunol. 11, 229-241, 1999

Potrykus etal., Mol. Gen. Genet., 199:183-188, 1985.

Potter et al., Proc. Nat'l Acad. Sci. USA, 81:7161-7165, 1984.

Queen and Baltimore, “Immunoglobulin gene transcription is activated by downstream sequence elements,” Cell, 35:741, 1983.

Quinn, Farina, Gardner, Krutzsch, and Levens, “Multiple components are required for sequence recognition of the ap1 site in the gibbon ape leukemia virus enhancer,” Mol. Cell. Biol., 9:4713, 1989.

Ravetch, J. V. & Lanier, L. L., Immune inhibitory receptors. Science 290, 84-89, 2000.

Redondo, Hata, Brocklehurst, and Krangel, “A T-Cell-Specific Transcriptional Enhancer Within. the Human T-Cell Receptor delta Locus,” Science, 247:1225, 1990.

Reisman and Rotter, “Induced expression from the moloney murine leukemia virus long terminal repeat during differentiation of human myeloid cells is mediated through its transcriptional enhancer,” Mol. Cell. Biol., 9:3571, 1989.

Resendez Jr., Wooden, and Lee, “Identification of highly conserved regulatory domains and protein-binding sites in the promoters of the rat and human genes encoding the stress-inducible 78-kilodalton glucose-regulated protein,” Mol. Cell. Biol., 8:4579, 1988.

Riddell et al., Restoration of viral immunity in immunodeficient humans by the adoptive transfer of T cell clones. Science, 257, 238-241, 1992.

Ridgeway, In: Rodriguez R L, Denhardt D T, ed. Vectors: A survey of molecular clonirig vectors and their uses. Stoneham: Butterworth, pp. 467-492, 1988.

Ripe, Lorenzen, Brenner, and Breindl, “Regulatory elements in the 5′ flanking region and the first intron contribute to transcriptional control of the mouse alpha-1-type collagen gene,” Mol. Cell. Biol., 9:2224, 1989.

Rippe etaL., Mol. Cell Biol., 10:689-695, 1990.

Rittling, Coutinho, Amarm, and Kolbe, “AP-1/jun-binding Sites Mediate Serum Inducibility of the Human Vimentin Promoter,” Nuc. Acids Res., 17:1619, 1989.

Rosen, Sodroski, and Haseltine, “The location of cis-acting regulatory sequences in the human t-cell lymphotropic virus type III (HTLV-111/LAV) long terminal repeat,” Cell, 41:813, 1988.

Roux et al., Proc. Nat'l Acad. Sci. USA, 86:9079-9083, 1989

Sakai, Helms, Carlstedt-Duke, Gustafsson, Rottman, and Yamamoto, “Hormone-Mediated Repression: A Negative Glucocorticoid-Response Element From the Bovine Prolactin Gene,” Genes and Dev., 2:1144, 1988.

Sambrook et al., Molecular Cloning: A Laboratory Manual, 2d Ed., Cold Spring Harbor Laboratory Press, Cold Spring Harbor, N.Y., 1989

Satake, Furukawa, and Ito, “Biological activities of oligonucleotides spanning the f9 point mutation within the enhancer region of polyoma virus DNA,” J. Virology, 62:970, 1988.

Schaffner, Schirm, Muller-Baden, Wever, and Schaffner, “Redundancy of Information in Enhancers as a Principle of Mammalian Transcription Control,” J. Mol. Biol., 201:81, 1988.

Searle, Stuart, and Palmiter, “Building a metal-responsive promoter with synthetic regulatory elements,” Mol. Cell. Biol., 5:1480, 1985.

Sharp and Marciniak, “HIV Tar: an RNA Enhancer?,” Cell, 59:229, 1989.

Shaul and Ben-Levy, “Multiple Nuclear Proteins in Liver Cells are Bound to Hepatitis B Virus Enhancer Element and its Upstream Sequences,” EMBO J., 6:1913, 1987.

Sherman, Basta, Moore, Brown, and Ting, “Class II Box Consensus Sequences in the HLA-DR.alpha. Gene: Transcriptional Function and Interaction with Nuclear Proteins,” Mol. Cell. Biol., 9:50 1989.

Sleigh and Lockett, “SV40 Enhancer Activation During Retinoic-Acid-Induced Differentiation of F9 Embryonal Carcinoma Cells,” J. EMBO, 4:3831, 1985.

Spalholz, Yang, and Howley, “Transactivation of a Bovine Papilloma Virus Transcriptional Regulatory Element by the E2 Gene Product,” Cell, 42:183, 1985.

Spandau and Lee, “Trans-Activation of Viral Enhancers by the Hepatitis B Virus X Protein,” J. Virology, 62:427, 1988.

Spandidos and Wilkie, “Host-Specificities of Papilloma Virus, Moloney Murine Sarcoma Virus and Simian Virus 40 Enhancer Sequences,” EMBO J., 2:1193, 1983.

Stephens and Hentschel, “The Bovine Papilloma Virus Genome and its Uses as a Eukaryotic Vector,” Biochem. J., 248:1, 1987.

Stuart, Searle, and Palmiter, “Identification of Multiple Metal Regulatory Elements in Mouse Metallothionein-I Promoter by Assaying Synthetic Sequences,” Nature, 317:828, 1985.

Sullivan and Peterlin, “Transcriptional Enhancers in the HLA-DQ Subregion,” Mol. Cell. Biol., 7:3315, 1987.

Swartzendruber and Lehman, “Neoplastic Differentiation: Interaction of Simian Virus 40 and Polyoma Virus with Murine Teratocarcinoma Cells,” J. Cell. Physiology, 85:179, 1975.

Takebe, Seiki, Fujisawa, Hoy, Yokota, Arai, Yoshida, and Arai, “SRα Promoter: An Efficient and Versatile Mammalian cDNA Expression System Composed of the Simian Virus 40

Early Promoter and the R-U5 Segment of Human T-Cell Leukemia Virus Type β Long Terminal Repeat,” Mol. Cell. Biol., 8:466, 1988.

Tavernier, Gheysen, Duerinck, Can Der Heyden, and Fiers, “Deletion Mapping of the Inducible Promoter of Human IFN-beta Gene,” Nature, 301:634, 1983.

Taylor and Kingston, “ElA Trans-Activation of Human HSP70 Gene Promoter Substitution Mutants is Independent of the Composition of Upstream and TATA Elements,” Mol. Cell. Biol., 10:176, 1990.

Taylor and Kingston, “Factor Substitution in a Human HSP70 Gene Promoter: TATA-Dependent and TATA-Independent Interactions,” Mol. Cell. Biol., 10:165, 1990a.

Taylor, Solomon, Weiner, Paucha, Bradley, and Kingston, “Stimulation of the Human Heat-Shock Protein 70 Promoter in vitro by Simian Virus 40 Large T Antigen,” J. Biol. Chem., 264:15160, 1989.

Temin, In: Gene Transfer, Kucherlapati (ed.), New York: Plenum Press, pp. 149-188, 1986

Thiesen, Bosze, Henry, and Charnay, “A DNA Element Responsible for the Different Tissue Specificities of Friend and Moloney Retroviral Enhancers,” J. Virology, 62:614, 1988.

Thompson et al., CD28 activation pathway regulates the production of multiple T-cell-derived lymphokines/cytokines. Proc. Nat'l Acad. Sci. USA 86, 1333-1337, 1989.

Tratschin, West, Sandbank, and Carter, “A human parvovirus, adeno-associated virus, as a eucaryotic vector: transient expression and encapsidation of the procaryotic gene for chloramphenicol acetyltransferase,” Mol. Cell. Biol., 4:2072-2081, 1984.

Treisman, “Transient Accumulation of c-fos RNA Following Serum Stimulation Requires a Conserved 5′ Element and c-fos 3′ Sequences,” Cell, 42:889, 1985.

Tronche, Rollier, Bach, Weiss, and Yaniv, “The Rat Albumin Promoter: Cooperation with Upstream Elements is Required When Binding of APF/HNF 1 to the Proximal Element is Partially Impaired by Mutation or Bacterial Methylation,” Mol. Cell. Biol., 9:4759, 1989.

Tronche, Rollier, Herbomel, Bach, Cereghini, Weiss, and Yaniv, “Anatomy of the Rat Albumin Promoter,” Mol Biol. Med., 7:173, 1990.

Trudel and Constantini, “A 3′ Enhancer Contributes to the Stage-Specific Expression of the Human Beta-Globin Gene,” Genes and Dev., 6:954, 1987.

Tsumaki et al., “Modular arrangement of cartilage- and neural tissue-specific. cis-elements in the mouse alpha2(XI) collagen promoter,” J. Biol Chem. 273(36):22861-22864, 1998.

Tur-Kaspa et al., Mol. Cell Biol., 6:716-718, 1986

Tyndall, La Mantia, Thacker, Favaloro, and Kamen, “A Region of the Polyoma Virus Genome Between the Replication Origin and Late Protein-Coding Sequences is Required in cis for Both Early Gene Expression and Viral DNA Replication,” Nuc. Acids. Res., 9:6231, 1981.

U.S. Pat. No. 4,196,265

U.S. Pat. No. 4,554,101

U.S. Pat. No. 4,683,202

U.S. Pat. No. 4,684,611

U.S. Pat. No. 4,797,368

U.S. Pat. No. 4,952,500

U.S. Pat. No. 5,139,941

U.S. Pat. No. 5,302,523

U.S. Pat. No. 5,322,783

U.S. Pat. No. 5,384,253

U.S. Pat. No. 5,440,013

U.S. Pat. No. 5,446,128

U.S. Pat. No. 5,464,765

U.S. Pat. No. 5,475,085

U.S. Pat. No. 5,538,877

U.S. Pat. No. 5,538,880

U.S. Pat. No. 5,538,880

U.S. Pat. No. 5,550,318

U.S. Pat. No. 5,550,318

U.S. Pat. No. 5,563,055

U.S. Pat. No. 5,580,859

U.S. Pat. No. 5,589,466

U.S. Pat. No. 5,591,616

U.S. Pat. No. 5,610,042

U.S. Pat. No. 5,610,042

U.S. Pat. No. 5,618,914

U.S. Pat. No. 5,656,610

U.S. Pat. No. 5,670,155

U.S. Pat. No. 5,672,681

U.S. Pat. No. 5,674,976

U.S. Pat. No. 5,702,932

U.S. Pat. No. 5,710,245

U.S. Pat. No. 5,736,524

U.S. Pat. No. 5,780,448

U.S. Pat. No. 5,789,215

U.S. Pat. No. 5,840,833

U.S. Pat. No. 5,859,184

U.S. Pat. No. 5,928,906

U.S. Pat. No. 5,929,237

U.S. Pat. No. 5,945,100

U.S. Pat. No. 5,981,274

U.S. Pat. No. 5,994,136

U.S. Pat. No. 5,994,624

U.S. Pat. No. 6,013,516

U.S. Pat. No. 5,925,565

U.S. Pat. No. 5,935,819

Vannice and Levinson, “Properties of the Human Hepatitis B Virus Enhancer: Position Effects and Cell-Type Nonspecificity,” J. Virol., 62:1305, 1988.

Vasseur, Kress, Montreau, and Blangy, “Isolation and Characterization of Polyoma Virus Mutants Able to Develop in Multipotential Murine Embryonal Carcinoma Cells,” Proc Nat'l Acad. Sci. USA, 77:1068, 1980.

Vita et al., “Novel miniproteins engineered by the transfer of active sites to small

Wagner et al., Science, 260:1510-1513, 1990.

Waldmann et al., Enhanced endothelial cytopathogenicity induced by a cytomegalovirus strain propagated in endothelial cells. J. Med. Virol., 28, 223-230, 1989.

Wang and Calame, “SV40 enhancer-binding factors are required at the establishment but not the maintenance step of enhancer-dependent transcriptional activation,” Cell, 47:241, 1986.

Weber, De Villiers, and Schaffner, “An SV40 ‘Enhancer Trap’ Incorporates Exogenous Enhancers or Generates Enhancers From its Own Sequences,” Cell, 36:983, 1984.

Weinberger, Jat, and Sharp, “Localization of a Repressive Sequence Contributing to B-cell Specificity in the Immunoglobulin Heavy-Chain Enhancer,” Mol. Cell. Biol., 8:988, 1984.

Wills et al., The human cytotoxic T-lymphocyte (CTL) response to cytomegalovirus is dominated by structural protein pp65: frequency, specificity, and T-cell receptor usage of pp65-specific CTL. J. Virol., 70, 7569-7579, 1996.

Wilson et al., “Implantation of vascular grafts lined with genetically modified endothelial cells,” Science, 244:1344-1346, 1989.

Winoto and Baltimore, “αβ-lineage-specific Expression of the α T-Cell Receptor Gene by Nearby Silencers,” Cell, 59:649, 1989.

WO 92/17198

WO 94/09699

WO 95/06128

Wong et al., “Appearance of β-lactamase activity in animal cells upon liposome mediated gene transfer,” Gene, 10:87-94, 1980.

Wu and Wu, “Evidence for targeted gene delivery to HepG2 hepatoma cells in vitro,” Biochemistry, 27:887-892, 1988.

Wu and Wu, “Receptor-mediated in vitro gene transfections by a soluble DNA carrier system,” J. Biol. Chem., 262:4429-4432, 1987.

Wu et al., “An activating immunoreceptor complex formed by NKG2D and DAP 10,” Science, 285, 730-732, 1999.

Wu et al., “Promoter-dependent tissue-specific expressive nature of imprinting gene, insulin-like growth factor II, in human tissues,” Biochem Biophys Res Commun. 233(1):221-226, 1997.

Yang et al., “In vivo and in vitro gene transfer to mammalian somatic cells by particle bombardment,” Proc. Nat'l Acad. Sci. USA, 87:9568-9572, 1990

Yutzey, Kline, and Konieczny, “An Internal Regulatory Element Controls Troponin I Gene Expression,” Mol. Cell. Biol., 9:1397, 1989.

Zhao-Emonet et al., “The equine herpes virus 4 thymidine kinase is a better suicide gene than the human herpes virus I thymidine kinase,” Gene Ther. 6(9):1638-1642, 1999.

1. A method for expanding a human T-cell population that expresses a natural or engineered NKG2D comprising contacting said population with an NKG2D ligand.

2. The method of claim 1, wherein the NKG2D ligand is an anti-NKG2D antibody, or an NKG2D-binding fragment thereof.

3. The method of claim 1, wherein said contacting is performed in vivo.

4. The method of claim 1, wherein said contacting is performed ex vivo.

5. The method of claim 1, wherein said cell population is a CD8⁺ population or a CD4⁺ population.

6. The method of claim 1, wherein said cell population is a T cell population, an NK cell population or a monocyte population.

7. The method of claim 6, wherein said cell population is T cell population.

8. The method of claim 7, wherein said T cell population is an antigen-specific T cell population.

9. The method of claim 6, wherein said T cell population is from a subject with a primed anti-tumor response.

10. The method of claim 6, wherein said T cell population is from a subject with a primed anti-viral response.

11. The method of claim 6, wherein said T cell population is from an immunocompromised subject.

12. The method of claim 6, wherein said T cell population also is induced to secrete lymphokines.

13. The method of claim 2, wherein said anti-NKG2-D antibody fragment thereof is selected from the group consisting of Fab, F(ab′)₂, and single-chain antibody.

14. A method for inducing lymphokine secretion from a human cell population that expresses a natural or engineered comprising contacting said population with an anti-NKG2-D antibody, or an NKG2-D-binding fragment thereof.

15. The method of claim 14, wherein said lymphokine is selected from the group consisting of INF-γ, TNF-α, GM-CSF, IL-2 and IL-4.

16. A method for enhancing an antigen-specific T cell response in a subject comprising (a) obtaining a population of antigen-specific T cells, (b) contacting said population of antigen-specific T cells with an anti-NKG2-D antibody, or an NKG2-D-binding fragment thereof, and (c) administering said population to said subject.

17. A method for treating cancer comprising (a) obtaining a population of antigen-specific T cells from a subject having cancer, (b) contacting said population of antigen-specific T cells with an anti-NKG2-D antibody, or an NKG2-D-binding fragment thereof, and (c) administering said population to said subject.

18. The method of claim 17, wherein said cancer is an epithelial tumor.

19. The method of claim 18, wherein said epithelial tumor is a carcinoma.

20. The method of claim 19, wherein said carcinoma is a carcinoma of the breast, lung, colon, kidney, prostate, or ovary.

21. The method of claim 17, wherein said cancer is a melanoma.

22. A method for treating a viral infection comprising (a) obtaining a population of antigen-specific T cells from a subject having a viral infection, (b) contacting said population of antigen-specific T cells with an anti-NKG2-D antibody, or an NKG2-D-binding fragment thereof, and (c) administering said population to said subject.

23. A method of stimulating the immune system of an immunocompromised subject comprising (a) obtaining a population of antigen-specific T cells from said subject, (b) contacting said population of antigen-specific T cells with an anti-NKG2-D antibody, or an NKG2-D-binding fragment thereof, and (c) administering said population to said subject.

24. A method of stimulating an effector function of a lymphocyte comprising (a) obtaining a population of lymphocytes, and (b) contacting said population of lymphocytes with an anti-NKG2-D antibody, or an NKG2-D-binding fragment thereof.

25. A method of stimulating a memory function of a lymphocyte comprising (a) obtaining a population of lymphocytes, and (b) contacting said population of lymphocytes with an anti-NKG2-D antibody, or an NKG2-D-binding fragment thereof.
